(19) United States
(12) Patent Application Publication
(10) Pub. No.: US 2026/0224499 A1
(43) Pub. Date: Aug. 6, 2026

(54) COMPOSITIONS AND METHODS FOR TREATING PULMONARY CONDITIONS

(71) Applicant: MAYO FOUNDATION FOR MEDICAL EDUCATION AND RESEARCH, Rochester, MN (US)

(72) Inventors: Atta Behfar, Rochester, MN (US); Skylar Alexis Rizzo, Rochester, MN (US); Robert Vassallo, Rochester, MN (US); Shaohua Wang, Rochester, MN (US)

(21) Appl. No.: 19/150,339

(22) PCT Filed: Feb. 2, 2024
(86) PCT No.: PCT/US2024/014217

§ 371 Date: Jul. 23, 2025

Related U.S. Application Data
(60) Provisional application No. 63/443,090, filed on Feb. 3, 2023.

(51) Int. Cl. A61K 9/50; A61K 9/00; A61K 38/44; A61P 11/00

(52) U.S. Cl. CPC A61K 9/5068; A61K 9/0078; A61K 38/44; A61P 11/00; C12Y 114/99003

(57) ABSTRACT

A method of treating a pulmonary condition in a subject having or at risk of having the pulmonary condition generally includes administering to the subject a therapeutic composition in an amount effective to treat the pulmonary′ condition. Generally, the therapeutic composition includes purified exosome product (PEP) exosomes and a pharmaceutically acceptable carrier. In one or more embodiments, the PEP exosomes are modified to include at least one exogenous active agent. In one or more embodiments, the therapeutic composition is formulated for delivery directly to a potion of tire pulmonary tract.

### CROSS-REFERENCE TO RELATED APPLICATION

This application claims the benefit of U.S. Provisional Patent Application No. 63/443,090, filed Feb. 3, 2023, which is incorporated herein by reference in its entirety.

### SEQUENCE LISTING

This application contains a Sequence Listing electronically submitted via EFS-Web to the United States Patent and Trademark Office as an XML file entitled “0560.000024WO01.xml” having a size of 8 kilobytes and created on Jan. 30, 2024. The information contained in the Sequence Listing is incorporated by reference herein.

### SUMMARY

This disclosure describes, in one aspect, a method of treating a pulmonary condition in a subject having or at risk of having the pulmonary condition. Generally, the method includes administering to the subject a therapeutic composition in an amount effective to treat the pulmonary condition. Generally, the therapeutic composition includes purified exosome product (PEP) exosomes and a pharmaceutically acceptable carrier.

In one or more embodiments, the PEP exosomes are spherical or spheroid exosomes having a diameter no greater than 300 nm.

In one or more embodiments, the composition includes from 1-10¹⁰ PEP exosomes to 1×10¹¹ PEP exosomes.

In one or more embodiments, the therapeutic composition is formulated for delivery directly to a portion of the subject's pulmonary tract. In one or more of these embodiments, the therapeutic composition is formulated for delivery directly to the lung bed.

In one or more embodiments, the therapeutic composition is nebulized.

In one or more embodiments, the therapeutic composition is administered in an amount effective to decrease lung compliance (Cst), decrease mean linear intercept (Lm), increase lymphocytic infiltrates in perivascular areas, increase proportion of CD4⁺ FOXP3⁺ Tregs in perivascular lymphocytes, increase proportion of CD22⁺ plasma cells in perivascular lymphocytes, decrease expression of inflammatory signaling pathway genes, decrease expression of fibrotic signaling genes, decrease expression of PD/PD-L1 cancer immunotherapy genes, decrease expression of tumor microenvironment pathway genes, decrease phagosome formation, decrease expression of tumor necrosis factor (TNF), decrease expression of IL-17A-regulated pathways, decrease immune cell chemotaxis, decrease expression of NF-κB signaling pathway genes, decrease expression of senescence pathway genes, increase expression of wound healing pathway genes, decrease S100A8⁺ macrophages in alveolar and/or interstitial regions of the lung, decrease S100A9⁺ macrophages in alveolar and/or interstitial regions of the lung, increase heme oxygenase-1 (HO-1) in a whole lung lysate, decrease NF-κB p65 in a whole lung lysate, decrease caspase 8 in a whole lung lysate, decrease caspase 3 in a whole lung lysate, decrease CXCL12 transcription, increase expression of an antioxidant by alveolar epithelial cells, decrease apoptosis of alveolar epithelial cells, or any combination of two or more of the foregoing, compared to a subject to which the PEP composition is not administered. In one or more of these embodiments, the antioxidant is SOD 1-3 or heme oxygenase (HO-1).

In one or more embodiments, the PEP exosomes contain an antioxidant compound. In one or more of these embodiments, the antioxidant compound is heme oxygenase (HO-1).

In one or more embodiments, at least a portion of the PEP exosomes are modified to include at least exogenous active agent. In one or more of these embodiments, the exogenous active agent includes a polypeptide or a nucleic acid. In one or more of these embodiments, the nucleic acid is an mRNA that encodes a therapeutic polypeptide or an inhibitory RNA.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Histogram display of PEP particle size and concentration measured in NanoSight.

FIG. 2. Representative Xenogen images of lungs and other internal organs of a mouse exposed to far red dye-labeled PEP via nebulization. (A) Xenogen image of the lungs, livers, stomachs, and intestines of mice nebulized with 5×10⁹ labeled PEP extracellular vesicles (EVs) daily for 15 days. (B) Internal organs were harvested and imaged in Xenogen IVIS Spectrum. The fluorescence scale bar in both (A) and (B) indicates the epi-fluorescence intensity of the PEP uptake was 2.5×10⁹ to 3.0×10⁹ PEP exosomes.

FIG. 3. Line graph of epifluorescence intensity measurement of accumulated labeled PEP uptake over time in the lungs of mice.

FIG. 4. Murine lung section acquired from a mouse exposed to PEP aerosol. (A) Normal saline control mouse lung showed no positive staining for human CD63 (a marker for PEP). (B) Perinuclear stain of CD63 (a protein marker of PEP, bright green) in type I alveolar epithelial cells (white arrows, red cell membrane staining for aquaporin 5). (C) Perinuclear stain of CD63 (protein serving as a marker of PEP, bright green) in type I alveolar epithelial cells (white arrows, red cell membrane staining for aquaporin 5). (D) Perinuclear stain of CD63 (a protein marker of PEP, bright green) in type I alveolar epithelial cells (white arrows, red cell membrane staining for aquaporin 5). (E) Perinuclear stain of CD63 (protein serving as a marker of PEP, bright green) in type II alveolar epithelial cells (“*” indicates red cytoplasm—surfactant protein C label). (F) Perinuclear stain of CD63 (a protein marker of PEP, bright green) in alveolar macrophages (“#” indicates green cytoplasm staining).

FIG. 5. Lung morphometric assessment. (A) Study design and time course indicate a total of four months duration of the study. PEP nebulization began three months after cigarette smoke exposure. (B) Bodyweight change indicates that chronic cigarette smoke exposure caused significant weight loss compared to room air exposed control mice (p<0.0001). In contrast, room air exposed negative control mice gained weight over time. High-dose PEP treatment at 15 mg/mL concentration reduced the bodyweight loss significantly (*P=0.029) compared to the cigarette smoke exposed placebo control group.

FIG. 6. Lung morphometric assessment. (A) Static lung compliance (Cst) measurement demonstrates a significant increase in the cigarette smoke-exposed placebo control group (****p<0.0001) compared to the room air-exposed sham controls. (B) Upward shift of the PV-curves in the cigarette smoke-exposed placebo control group (****p<0.0001) compared to the room air-exposed sham controls. PEP treatment significantly reduced this cigarette smoke-induced increase in static lung compliance at both dosages (**p=0.009 for low-dose PEP, and *p=0.018 for high-dose PEP).

FIG. 7. Representative images of hematoxylin and eosin (H&E)-stained lung sections. (A) Negative control. (B) cigarette smoke-exposed placebo control. (C) cigarette smoke-exposed, 7.5 mg/mL PEP. (D) cigarette smoke-exposed, 15 mg/mL PEP.

FIG. 8. Lung morphometric assessment. (A) The lung macrophage (Lm) count in the cigarette smoke-exposed placebo control group (NS/CS. CTL) was significantly less than the room air normal control group (****p<0.0001). In contrast, the Lm count was improved considerably in the low-dose PEP (***p=0.0004) and high-dose PEP groups (*p=0.042) compared to the cigarette smoke-exposed placebo controls. (B) Increased lymphocytic infiltrates were observed in the perivascular regions of the lungs exposed to the cigarette smoke compared to the room air control mice (*p=0.0²⁴). High-dose PEP nebulization in the cigarette smoke-exposed mice further increased the lymphocytic infiltrates in these regions compared to the cigarette smoke-exposed placebo control mice (*p=0.0232). This increased lymphocytic infiltrate was also seen in PEP nebulized lungs of room air control mice compared to the room air control mice without PEP nebulization (p<0.0001). (C) PEP treatment in normal control mice and cigarette smoke-exposed mice did not cause a significant increase in the thickness of the small arterial wall compared to the normal room air control mice or cigarette smoke-exposed placebo control mice.

FIG. 9. Transcriptomic RNA-Seq data analysis. Heatmap of targeted immuno-oncology mouse panel gene set reveals distinct gene expression profiles among the experimental groups.

FIG. 10. Transcriptomic RNA-Seq data analysis. (A-C) Unbiased GSEA analysis of gene sets in the cigarette smoke-exposed mouse lung tissues compared to the control lungs exposed to the room air. (A) Apoptosis signaling pathways. (B) DNA repair. (C) inflammatory response signaling. (D-F) High-dose PEP nebulization (15 mg/mL) in cigarette smoke-exposed mice partially reversed this enrichment of the gene sets in these pathways compared to the cigarette smoke-exposed placebo control mice, although the false discovery rates (FDR) did not reach the cut-off value of 0.25. (D) Apoptosis signaling pathways. (E) DNA repair. (F) inflammatory response signaling.

FIG. 11. Summary of top canonical pathways generated from paired core analysis of Ingenuity Pathway Analysis (IPA). In the PEP15/CS versus NS/CS_CTL paired comparison, PEP nebulization reversed some upregulated or downregulated pathways by cigarette smoke exposure.

FIG. 12. Characterization of the lymphocytic infiltrates. (A) Representative image of the lymphocytic infiltrate indicates CD4⁺ T (red fluorescence) and CD20⁺ B (green fluorescence) lymphocytes origin of the cell composition. (B) Representative low power images of CD4⁺FOXP3⁺ Treg cells among CD4⁺ T lymphocytes within the lymphocytic infiltrates. (C) Representative high-power images of CD4⁺FOXP3⁺ Treg cells among CD4⁺ T lymphocytes within the lymphocytic infiltrates. (D) Increased proportion of FOXP3⁺CD4⁺/CD4⁺ T cells was seen in high-dose (15 mg/mL) PEP nebulized lungs compared to the cigarette smoke-exposed placebo control lungs (p=0.04). (E) Representative low power image of CD22⁺ (green fluorescence) plasma cells among total lymphocytic infiltrating cells. (F) Representative low power image of CD22⁺CD19⁺ plasma cells among total lymphocytic infiltrating cells. (G) Representative low power image of CD22⁺CD138⁺ plasma cells among total lymphocytic infiltrating cells. Scale bars in (A), (B), (E), and (F) represent 50 microns (μm), scale bars in (C) and (G) represent 20 μsm.

FIG. 13. Representative images of lung macrophages stained as indicated. (A) S100A9. (B) CD206. (C) S100A9 and CD206 merged. (D) S100Ab and CD206. (E) Representative images and quantification of S100A9 positively stained macrophages in sham control lungs. (F) Representative images and quantification of S100A9 positively stained macrophages in cigarette smoke-exposed placebo control lungs. (G) Representative images and quantification of S100A9 positively stained macrophages in cigarette smoke-exposed lungs treated with nebulized PEP (15 mg/mL). (H) Quantification of increased S10A9 positively stained cells in the cigarette smoke-exposed placebo control lungs from 54±4 (mean±SE) to 80±7 cells/image field (20×) (p<0.001). PEP nebulization at 15 mg/mL dosage markedly reduced this cigarette smoke-induced increase in S100A8/A9 expressing macrophages from 80±7 to 36±3 cells/image field (20×) for S100A9 (p<0.001). (I) Representative images and quantification of S100A8 positively stained macrophages in sham control lungs. (J) Representative images and quantification of S100A8 positively stained macrophages in cigarette smoke-exposed placebo control lungs. (K) Representative images and quantification of S100A8 positively stained macrophages in cigarette smoke-exposed lungs treated with nebulized PEP (15 mg/mL). (L) Quantification of increased S100A8 positively stained cells in the cigarette smoke-exposed placebo control from 59±5 to 89±5 cells/image field (20×) (p<0.001). PEP nebulization at 15 mg/mL dosage markedly reduced this cigarette smoke-induced increase in S100A8/A9 expressing macrophages from 89±5 to 46±3 cells/image field (20×) for S100A8 (p<0.001) compared to cigarette smoke controls. Scale bars in (A)-(C) represent 20 μm; scale bars in (E)-(G) and (I)-(K) represent 50 μm.

FIG. 14. Western blot and qPCR of whole mouse lung lysate to examine oxidative stress, NF-kB, and apoptosis signaling pathways.

FIG. 15. (A) cigarette smoke exposure increased the expression of HO-1 (*p=0.019), and PEP nebulization increased HO-1 expression in cigarette smoke-exposed mice lungs at both concentrations (****p<0.0001). (B) cigarette smoke exposure activated NF-κB signaling (p-NF-κB/TNF-κB) (**p=0.003), and PEP treatment decreased NF-κB activation at both concentrations (****p<0.0001). (C) cigarette smoke exposure also led to a decrease in the antiapoptotic molecule BCL2L2 (*p=0.043), and PEP treatment significantly increased BCL2L2 expression at both concentrations (****p<0.0001). (E) PEP treatment decreased pro-apoptotic caspase 8 (*p=0.034 for low-dose PEP and ***p<0.001 for high-dose PEP). (F) PEP treatment decreased pro-apoptotic caspase 3 (*p=0.042). (F) qPCR quantitative analysis shows that CXCL12 gene expression was increased by cigarette smoke exposure (*p=0.01) and decreased by PEP treatment at both low-dose (*p=0.025) and high-dose (p=0.002) concentrations. (G) Caspase 3 gene expression was increased after cigarette smoke chronic exposure (***p<0.001), and this cigarette smoke-induced increase was reversed after high-dose PEP treatment (15 mg/mL) (***p=<0.001). (H) Caspase 8 gene expression was increased after cigarette smoke chronic exposure (**p=0.004), and this cigarette smoke-induced increase was reversed after high-dose PEP treatment (15 mg/mL)(***p=<0.001).

FIG. 16. Anti-apoptotic effects of PEP. (A) Western blot of PEP from three separate batches shows highly enriched antioxidants SODs and HO-1. CD63 is a PEP marker used as internal control as the alpha-tubulin. (B) Live-cell imaging of DiR fluorescent-labeled PEP uptake 45 minutes after coincubation with cultured ATI cells. Fluorescent-labeled PEP was localized to the perinuclear region of the cells. PEP internalization peaked 45 minutes after internalization started. (C) Live-cell imaging of DiR fluorescent-labeled PEP uptake 45 minutes after coincubation with cultured ATII cells. Fluorescent-labeled PEP was localized to the perinuclear region of the cells. PEP internalization peaked 45 minutes after internalization started. (D) Apoptosis in vitro assay performed in IncuCyte indicates that PEP preincubation significantly decreased cigarette smoke extract-induced apoptotic cell death in ATII cells from at both low (3.75 mg/mL) and high (7.5 mg/mL) dosages (p<0.001). Apoptotic cells were identified by fluorescence-labeled caspase 3′/caspase 7′ cells. (E) Apoptosis in vitro assay performed in IncuCyte indicates that PEP preincubation significantly decreased cigarette smoke extract-induced apoptotic cell death in ATI cells from at both low (3.75 mg/mL) and high (7.5 mg/mL) dosages (p<0.001). Apoptotic cells were identified by fluorescence-labeled caspase 3′/caspase 7′ cells.

FIG. 17. Anti-apoptotic effects of PEP. (A) Western blot on primary rat ATII and ATI cells shows cigarette smoke exposure induced cellular antioxidative response to cigarette smoke extract stimulation. As shown in the figure, cigarette smoke exposure increased HO-1 expression, and this was further increased by preincubating the cells with PEP at both concentrations. (B) Quantitation of increase expression of HO-1 in ATIH cells (**p<0.01, ***p<0.001, ****p<0.0001). (C) Quantitation of increase expression of HO-1 in ATI cells (*p<0.05, **p<0.01, ***p<0.001l).

FIG. 18. Pulmonary exosome delivery procedures. (A) A schematic depicting the methods employed for pulmonary delivery of exosomes, including nebulization, intravenous, and pulmonary artery (PA) balloon catheter-guided approaches. (B) Photograph demonstrating jet nebulizer (red arrow) attachment to endotracheal tubing. (C) Fluoroscopy image showing placement of endotracheal tube (red arrow) in the trachea above the carina to allow bilateral pulmonary delivery. (D) Pulmonary angiogram of the right hung. (E) Angiogram demonstrating occlusion by balloon catheter of pulmonary artery branch with contrast injection and dashed box indicating area of zoomed in image in FIG. 18F. (F). Zoomed in image from FIG. 18E with arrow indicating area of balloon catheter occlusion (red arrow).

FIG. 19. Pulmonary absorption of exosomes with intravenous, PA, balloon catheter-guided, and nebulized delivery. (A) Xenogen imaging of uptake of DiR labeled PEP in the lungs for intravenous, PA balloon catheter-guided, and nebulized delivery. (B) Western blots demonstrating presence of PEP in lung tissue compared to PEP and control lung tissue (Ctrl) using exosomal proteins CD63 (green) and loading control actin (red). (C) Quantification of mean fluorescent signal (+/−SEM) of CD63 normalized to actin loading control. Dotted line represents level of control (Ctrl). (n=3).

FIG. 20. Xenogen imaging of uptake of DiR labeled PEP in the heart, liver, spleen, and kidney. Images of entire organ and organ sections are shown.

FIG. 21. Off-target absorption of exosomes with intravenous, PA balloon catheter-guided, and nebulized delivery. (A) Western blots demonstrating presence of PEP in liver compared to PEP and control organ tissue (-ctrl) using exosomal protein CD63 (green) and loading control GAPDH (green). (B) Quantification of mean fluorescent signal (+/−SEM) of CD63 normalized to GAPDH loading control. Level of control liver tissue shown in dotted line. (n=3) D-F. (C-E) Western blots demonstrating presence of PEP in heart (C), spleen (D), and kidney (E) compared to PEP and control organ tissue (-ctrl) using exosomal protein CD63 (green) and loading controls actin (red) or GAPDH (green).

FIG. 22. Off-target absorption of exosomes with intravenous, PA, balloon catheter-guided, and nebulized delivery. (A) Xenogen imaging of DiR labeled PEP uptake in the esophagus (white arrowhead) and trachea (white arrow) of control tissue compared to tissue exposed to nebulized PEP (top). (B) Endotracheal (ET) tube demonstrating loss of PEP on plastic tubing also shown (bottom). (C) Western blot demonstrating PEP uptake in the esophagus by nebulization compared to PEP and control pig esophagus tissue (-ctrl) with exosome marker CD63 (green) and GAPDH (green) loading control. (D) Western blot demonstrating PEP uptake in the trachea by nebulization compared to PEP and control pig trachea tissue (-ctrl) with exosome marker CD63 (green) and GAPDH (green) loading control. (E) Quantification of mean fluorescent signal (+/−SEM) of CD63 normalized to GAPDH loading control for trachea and esophagus with nebulization. The level of control esophagus or trachea tissue is shown with the labeled dotted lines. (n=3).

FIG. 23. Off-target absorption of exosomes with intravenous, PA, balloon catheter-guided, and nebulized delivery. (A) immunohistochemical staining of lung tissue with CD63 (brown) with hematoxylin counterstain (blue). Scale bar represents 100 μm. (B) Mean (+/−SEM) ratio of CD63 area to total lung tissue area in untreated (control), intravenous, PA balloon catheter-guided, or nebulization delivery methods. (n=9).

### DETAILED DESCRIPTION OF ILLUSTRATIVE EMBODIMENTS

This disclosure describes compositions and methods for treating a subject having, or at risk of having, a pulmonary condition. Generally, the compositions include purified exosome product (PEP) exosomes and a pharmaceutically acceptable carrier. The methods generally include administering the composition to a subject in an amount effective to treat the pulmonary condition, in one or more embodiments, the method may involve administering a nebulized formulation of the composition directly to tissues of the subject's pulmonary tract.

Chronic obstructive pulmonary disease (COPD) is characterized by chronic inflammation of small airways, thickening of airway walls, and progressive obstruction and destruction of alveolar units. Several mechanisms are involved in the development of the disease, including an influx of inflammatory cells into the lungs that lead to chronic inflammation, an imbalance between oxidative stress and antioxidative activity, an enhanced predisposition to infection, which further propagates inflammation, and the dysregulation of cellular apoptosis and regeneration in the lung. Repetitive alveolar and airway epithelial injury predispose a smoker to develop COPD. Injury induced by the repetitive inhalation of cigarette smoke (CS) and other environmental insults augment the pro-oxidative environment in epithelial cells that overwhelms endogenous protective mechanisms. This is further exacerbated by other insults such as viral and other lung infections. A repetitive cycle of acute and chronic oxidative stress on the airway and distal lung epithelial barrier eventually leads to maladaptive tissue response and persistent immune cell recruitment, further exacerbating the regional oxidative burden and stress on epithelial cells and resulting in a chronic cycle of persistent inflammation, maladaptive tissue regenerative responses, and eventually remodeling and loss of normal structure and function.

COPD causes considerable morbidity and premature mortality. There is an urgent need for new therapeutic approaches that not only alleviate symptoms but also restore function and homeostasis in the airways and alveolar spaces. Extracellular vesicles (EVs) are cell-secreted organelles used for intercellular communication. EVs carry a variety of molecular cargo that can influence a wide range of biological processes in recipient cells.

This disclosure describes compositions and methods for treating pulmonary conditions using extracellular vesicles. In one or more embodiments, the extracellular vesicles can include a purified, platelet-derived extracellular vesicle product referred to herein as PEP. PEP is fully characterized and methods for preparing PEP are described in International Patent Application No. PCT/US2018/065627 (published as International Publication No. WO 2019/118817), U.S. Patent Publication No. 2021/0169812 A1, and U.S. Pat. No. 10,596,123, each of which is incorporated by reference herein in its entirety. Briefly, PEP is a purified exosome product prepared using a cryodesiccation step that produces a product having a structure that is distinct from exosomes prepared using conventional methods. For example, PEP typically has a spherical or spheroidal structure and an intact lipid bilayer rather than a crystalline structure that results from the reaggregation of lipids of the exosome lipid bilayer after exosomes are disrupted during convention exosome preparation methods. The spherical or spheroid exosome structures generally have a diameter of no more than 300 nanometers (nm). Typically, a PEP preparation contains spherical or spheroid exosome structures that have a relatively narrow size distribution.

In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

In one or more embodiments, PEP may be modified to include one or more exogenous active agents. As used herein, the term “exogenous” refers to material that is not natively present in the PEP exosomes. Because PEP may be prepared from various starting materials, an active agent may be “exogenous” for PEP prepared from one source material even though it may be endogenous—i.e., natively present—in PEP exosomes prepared from another source. Thus, the evaluation of whether an active agent is exogenous depends on the source material used to prepare PEP. Exemplary exogenous active agents include, but are not limited to, a nucleic acid or a polypeptide. Methods for transforming extracellular vesicles and exemplary exogenous active agents are described in detail in US Patent Application Publication No. US 2021/0259969 A1 and in international Patent Application No. PCT/US2019/043172, which published as International Publication No. WO 2020/023594.

PEP has been shown to have efficacy in promoting wound healing (International Patent Application No. PCT/US2022/047721). PEP products are enriched with antioxidant enzymes, immunomodulatory molecules, and regenerative factors (US Patent Application Publication No. US 2022/241325 A 1; U.S. Provisional Patent Application No. 63/313,579, filed Feb. 24, 2022). This disclosure describes the use of PEP to directly deliver exosome cargo to alveolar epithelial cells and macrophages, promote antioxidant and anti-inflammatory capacity in the lung, and suppress cigarette smoke-induced oxidative injury and emphysematous changes in murine lungs.

Nebulization Effectively Delivers PEP into the Alveolar Regions of Spontaneously Breathing Mice

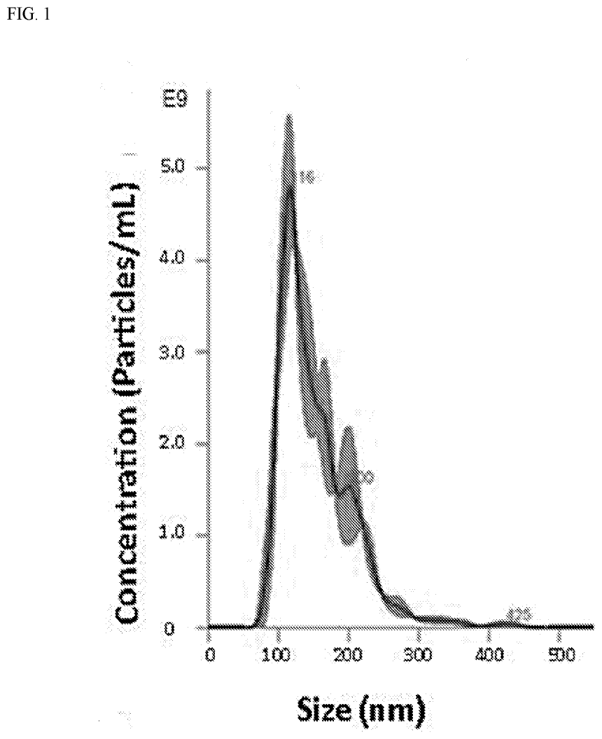

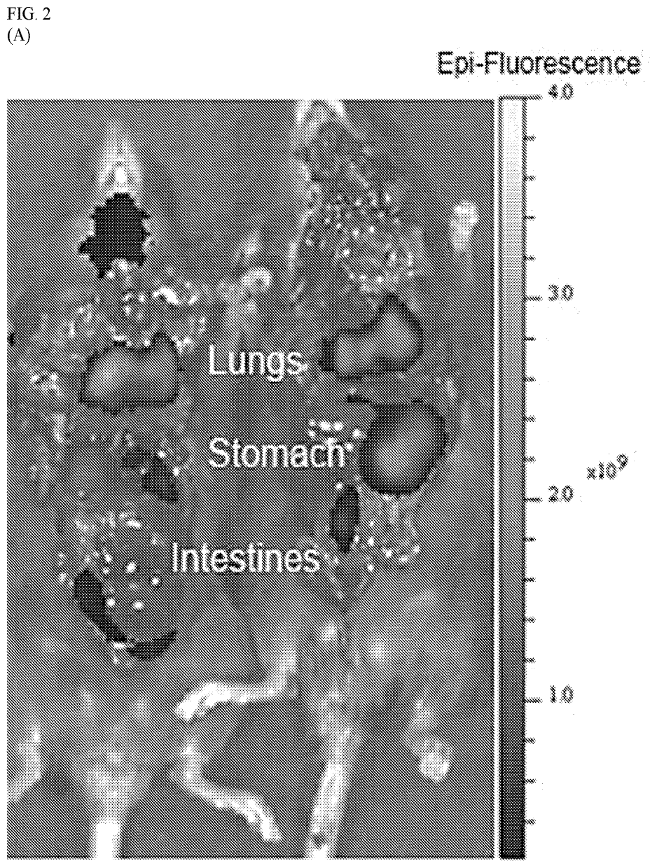

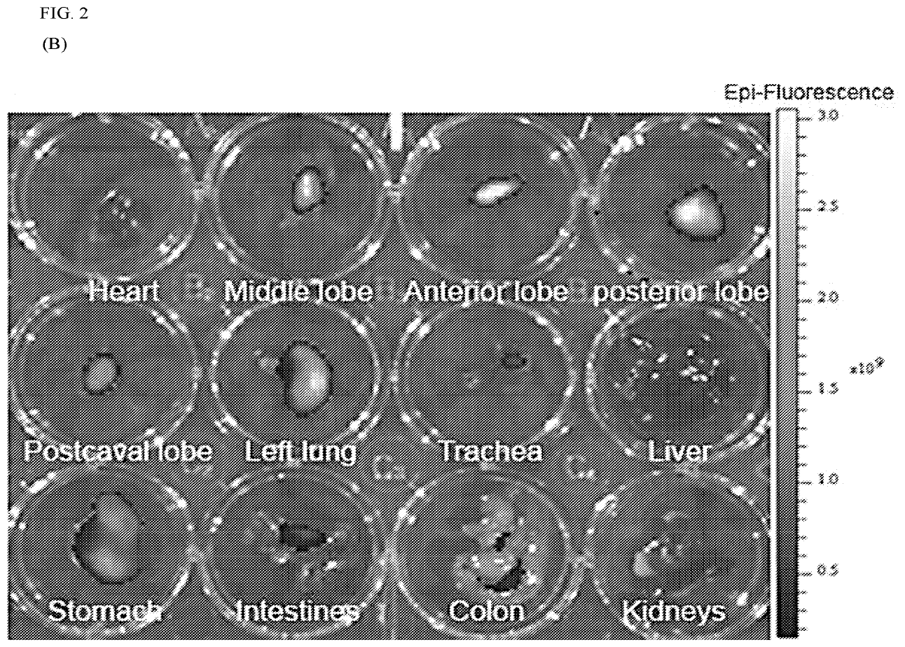

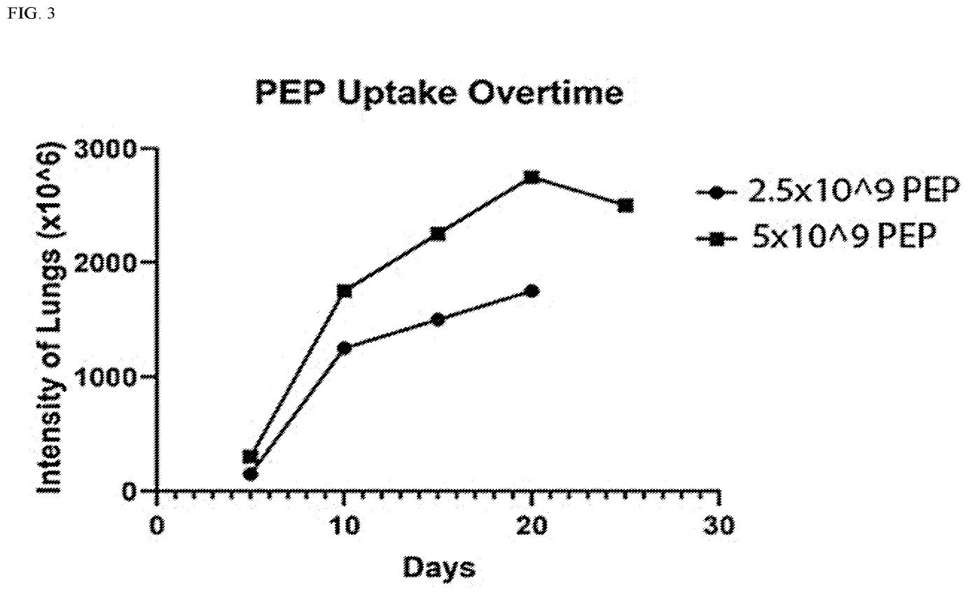

To determine the optimal route of PEP delivery with the primary goal of achieving PEP uptake by alveolar epithelial cells, carbocyanine DiOC18 (DiR) dye-labeled PEP was delivered by nebulization to spontaneously breathing non-sedated mice. The size of DiR-labeled PEP was determined to be 100 nm to 200 nm, and the concentrations used for nebulization were 2.5×10⁹ and 5×10⁹ EVs/mL (FIG. 1). The biodistribution of the inhaled DiR-PEP was examined using an in vivo imaging system. The highest epifluorescence was observed in all five lobes of the lungs with a peak value around 2.5×10⁹ to 3.0×10⁹ for higher dose PEP (5×10⁹ EVs/mL). This peak occurred 20 days following the start of nebulization, confirming that the lung was the major target site for nebulized PEP delivery (FIG. 3). There was no recorded PEP uptake identified in the heart, liver, spleen, or kidneys, although DiR-PEP was identified in the gastrointestinal tract with epifluorescence intensity around 1.5×10⁹ to 2×10⁹ in the stomach and 0.5×10⁹ in the intestines, presumably through the swallowing of excess PEP in the oral and nasal passages during nebulization (FIG. 2). PEP uptake in the lungs was cumulative until a peak was achieved at 20 days, and the fluorescence intensity of DiR-PEP increased over time in a dose-dependent manner (FIG. 3).

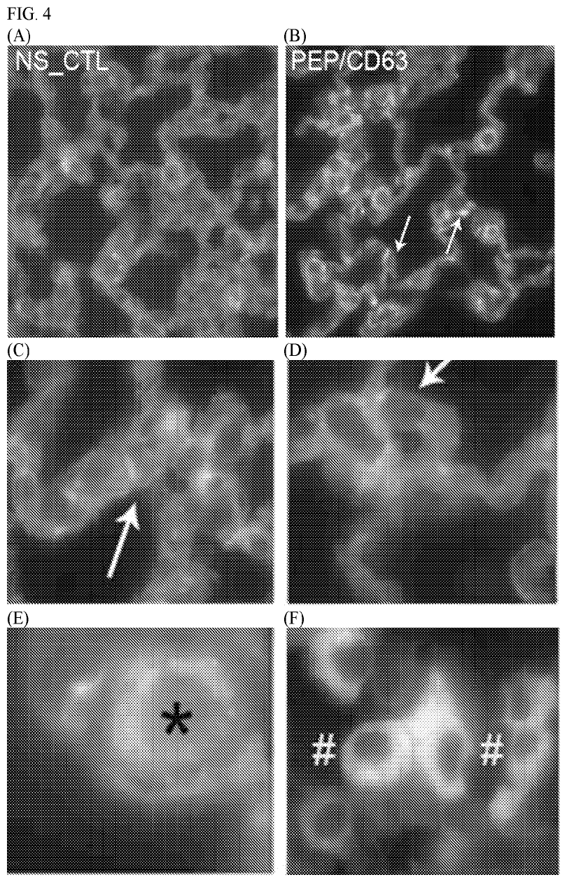

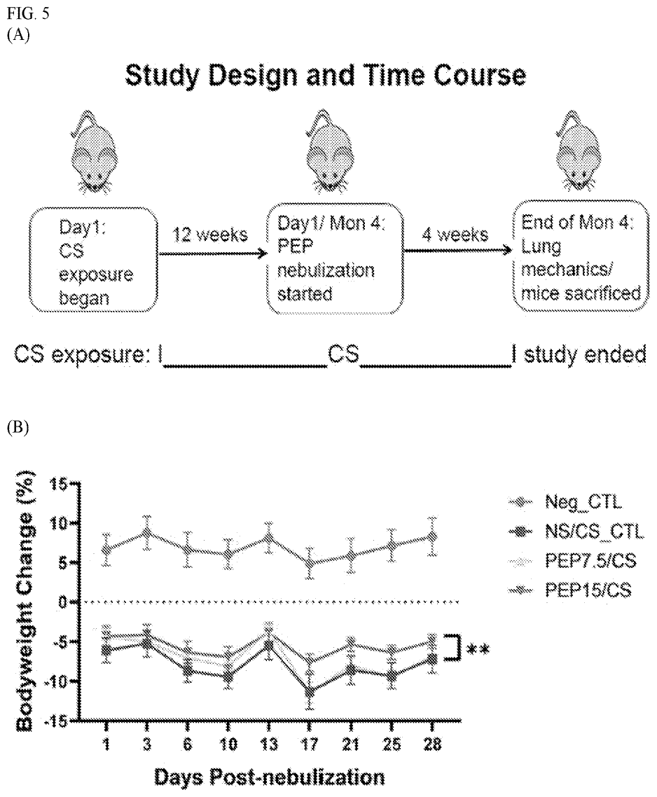

Further examination of the uptake of PEP cargo using immunohistostaining for human CD63, a PEP marker, revealed uptake of PEP contents in both type 1 and 2 alveolar epithelial cells and alveolar macrophages (FIG. 4). These data support nebulization of PEP as a suitable delivery mechanism to enable PEP delivery to the distal units of the lung—e.g., alveolar epithelial cells.

### PEP Mitigates Chronic Cigarette Smoke-Induced Murine Emphysema

To determine whether PEP can mitigate cigarette smoke effects and diminish the severity of cigarette smoke-induced emphysema, a cigarette smoke-induced mouse emphysema model was used. Treatment with nebulized PEP was initiated following 12 weeks of cigarette smoke inhalation. This experimental design was intentionally chosen to model a therapeutic approach to cigarette smoke-injury rather than a prophylactic approach. However, the use of PEP as a prophylactic approach for treating CS-induced lung injury or lung damage suffered from other forms of toxin exposure is a feasible therapeutic approach. Two escalating dosages of PEP were delivered by nebulization in the final four weeks of die experiment when the emphysematous change had already been initiated (FIG. 5A). Compared to control mice exposed to room air, mice exposed to chronic cigarette smoke inhalation weighed significantly less, suggesting a systemic effect of chronic cigarette smoke exposure, including loss of 5% to 10% of their original body weight (FIG. 5B). In contrast, room air-exposed control mice gained 5% to 10% of their initial body weight. PEP nebulization significantly reduced cigarette smoke-induced weight loss (p=0.029), with a more significant effect at the higher dose (15 mg/mL), indicating a dose-dependent systemic effect of PEP in chronic cigarette smoke exposed mice.

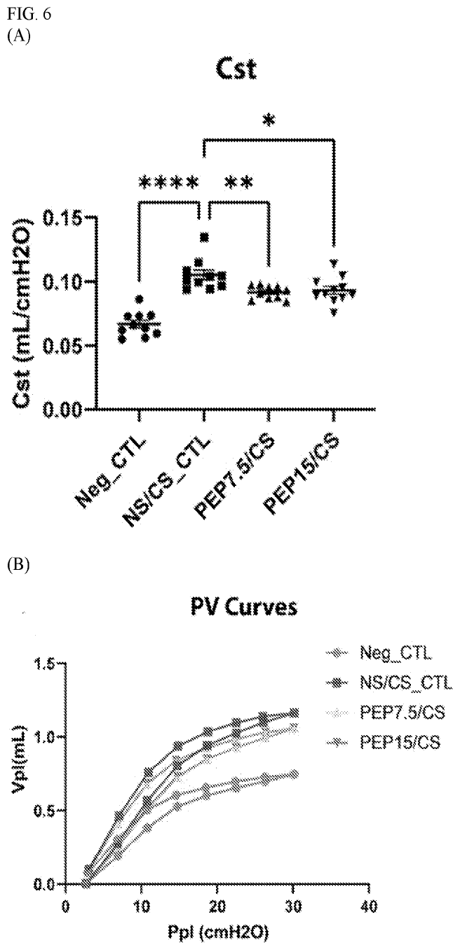

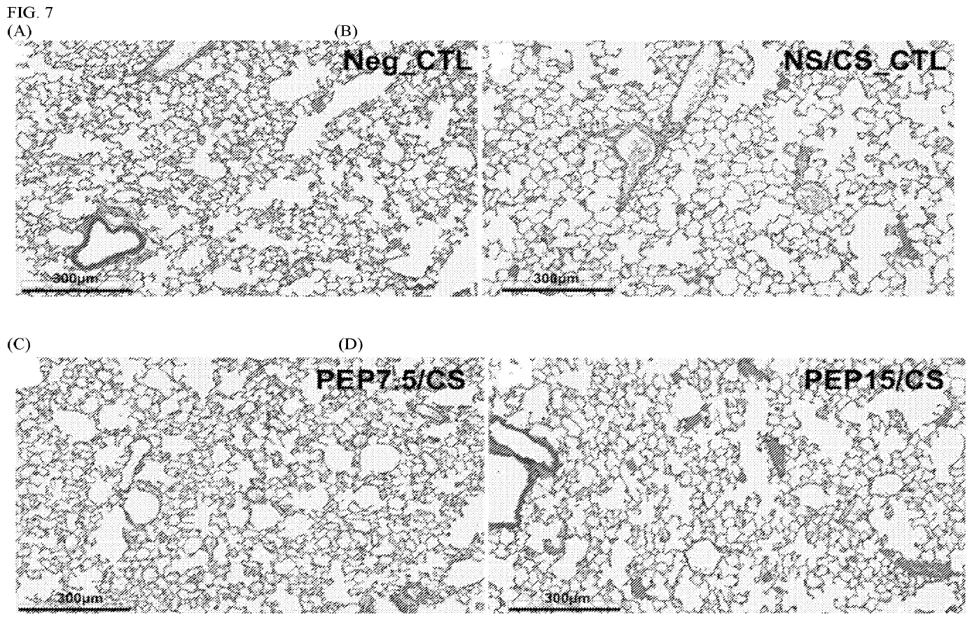

PEP nebulization also attenuated the cigarette smoke-induced increase in static lung compliance observed in mice that received only cigarette smoke. Cigarette smoke-exposed mice had higher lung compliance when compared with room air control mice. Static compliance (Cst) measured four months after the initiation of cigarette smoke exposure demonstrated a significant loss in the recoil force of the lung parenchyma reflected by the increase in Cst from 0.067±0.003 in the room air control mice (N=10) to 0.105±0.004 in the cigarette smoke-exposed mice (N=10) (p<0.001), leading to an upward shift of the PV-curve (FIG. 6). This increase in lung compliance is consistent with the induction of emphysema by cigarette smoke. PEP nebulization in cigarette smoke-exposed mice significantly attenuated this increase in lung compliance (or loss of recoil force) from 0.105±0.004-to 0.092±0.002 at low-dose PEP (7.5 mg/mL) (N=10)(p=0.006) and 0.093±0.003 at high-dose PEP (15 mg/mL)(N=11)(p=0.02), suggesting that both PEP concentrations were effective in mitigating cigarette smoke-induced loss of lung elastic recoil (FIG. 6).

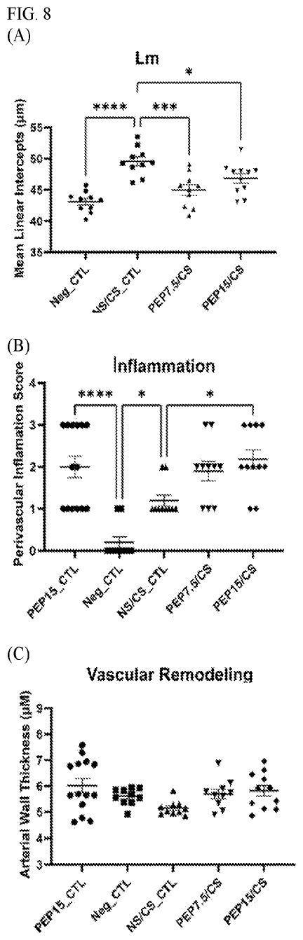

To determine the impact of PEP nebulization on alveolar anatomy, the mean linear intercept (Lm) length was measured to estimate the mean free distance of alveolar space. The Lm length was increased in mice exposed to only cigarette smoke, but this was significantly reduced by PEP treatment (FIG. 8A). The assessment of Lm is consistent with the observed effect of PEP treatment on lung compliance in cigarette-smoke-exposed mice and confirms that PEP nebulization reduced the cigarette smoke effect on alveolar size. A significant increase in Lm was seen in cigarette smoke-exposed placebo control mice compared to the room air negative control mice (p<0.001). In the cigarette-smoke-exposed mice that received PEP, the Lm was reduced in both low-dose PEP (p<0.001) and high-dose PEP (p=0.042) groups compared to the cigarette-smoke-exposed placebo control mice. This Lm result, together with the Cst measurement, demonstrates the effectiveness of PEP in alleviating the progression of murine emphysema.

Assessment of vascular structures did not reveal vascular remodeling or apparent differences in the small arterial wall thickness in mice that received PEP treatment only versus those that received room air only (FIG. 8C). Cigarette smoke exposure and PEP nebulization in cigarette-smoke-exposed mice also did not affect the arterial wall thickness. Studies on the histopathology of other internal organs besides lungs did not show any evidence of systemic toxicity, and the mice tolerated the inhaled PEP very well. To further ascertain the safety of nebulized PEP, an additional control group for PEP nebulization as a sole intervention was designed to examine the specificity and toxicity of PEP on normal control mice. Analysis of mice from this control group exposed only to nebulized PEP (15 mg/mL) showed no difference between PEP-nebulized room air control mice and the room air normal control mice in lung compliance and mean linear intercept. Histopathology review of the lungs and other internal organs revealed no abnormalities and no different from the normal control mice.

### PEP Nebulization Reverses Cigarette-Smoke-Induced Inflammatory Signaling Pathways

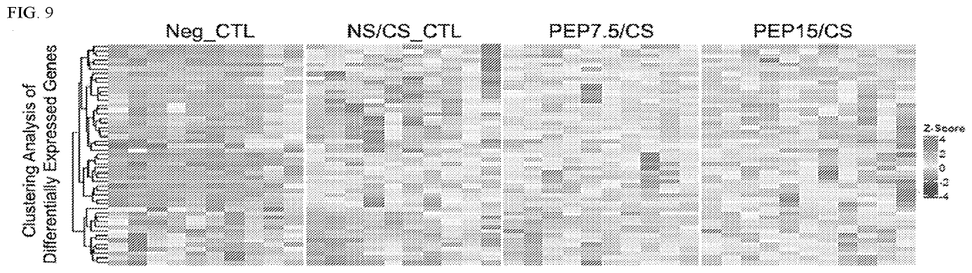

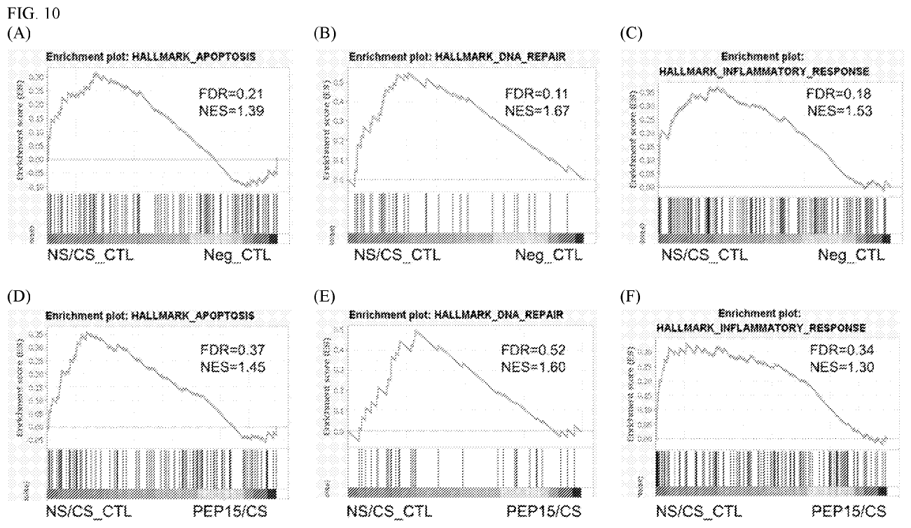

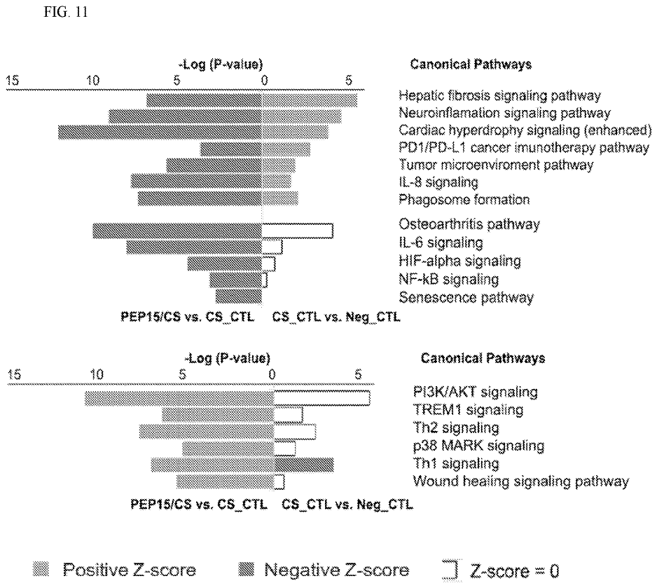

Following morphometric analysis of the mouse lung tissue for the phenotypic comparison among experimental groups, further transcriptomic studies were conducted to elucidate the underlying molecular mechanisms by which PEP prevented some of morphometric changes associated with progression in the model. Mouse lung tissue sections were sequenced using a commercially available mouse immuno-oncology gene panel that is not comprehensive but includes many of the genes involved in inflammation, immune cell trafficking, apoptosis, fibrosis and remodeling, and other processes pertinent to the pathogenesis of COPD. The heat map generated revealed three distinct gene expression profiles among the experimental groups (FIG. 9); room air control mice, cigarette-smoke-exposed placebo control mice, and PEP nebulized cigarette-smoke-exposed mice. There was no apparent distinction between the two PEP treated groups. Unbiased Gene Sets Enrichment Analysis (GSEA) revealed enrichment of gene sets in the apoptosis pathway (FDR=0.21, NES=1.39), DNA-repair pathway (FDR=0.11, NES=1.67), and inflammatory response pathway (FDR=0.18, NES=1.53) in cigarette smoke-exposed mice compared to the room air control mice (FIG. 10A-C). These enriched gene sets in these three pathways were attenuated by PEP treatment (15 mg/mL), although FDR's false discovery rate was higher than the typical cutoff value of 0.25 (FIG. 10D-F).

Further analysis of this gene data set using Ingenuity Pathway Analysis (IPA) revealed activation of inflammatory signaling pathways, fibrotic signaling, PD1/PD-L1 cancer immunotherapy and tumor microenvironment pathways, and increased phagosome formation in cigarette smoke-exposed mice compared to control mice (FIG. 11). Downregulation of the same processes was found in PEP-treated mice (FIG. 11). Cigarette smoke exposure induced an increase in the activity of upstream pro-inflammatory cytokines such as IL-1, IL-6, IL-17A, and TNF. Through these upstream regulators, cigarette smoke exposure activated immune cell responses, chemotaxis of phagocytes, and STAT3 signaling pathways. PEP nebulization in the cigarette smoke-exposed mice downregulated TNF and IL-17A regulated pathways and immune cell chemotaxis. PEP nebulization also downregulated NF-κB signaling and senescence pathway and upregulated wound healing pathway (FIG. 11). This result suggests that inhibiting inflammation, fibrogenesis, and senescence by PEP treatment may counteract the pathogenic mechanisms of cigarette smoke-induced emphysema in the lung.

### PEP Nebulization Increases the Proportion of Treg Cells and Suppresses Cigarette Smoke-Induced Lung Macrophage Recruitment

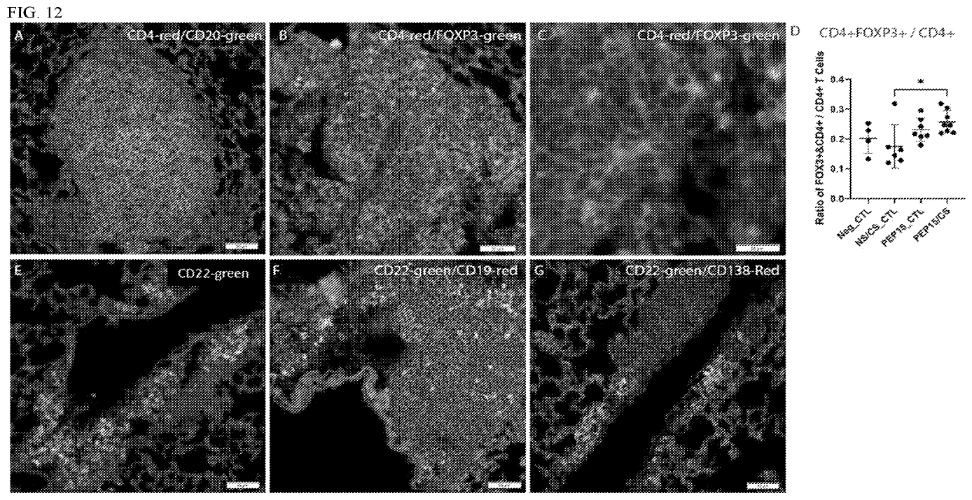

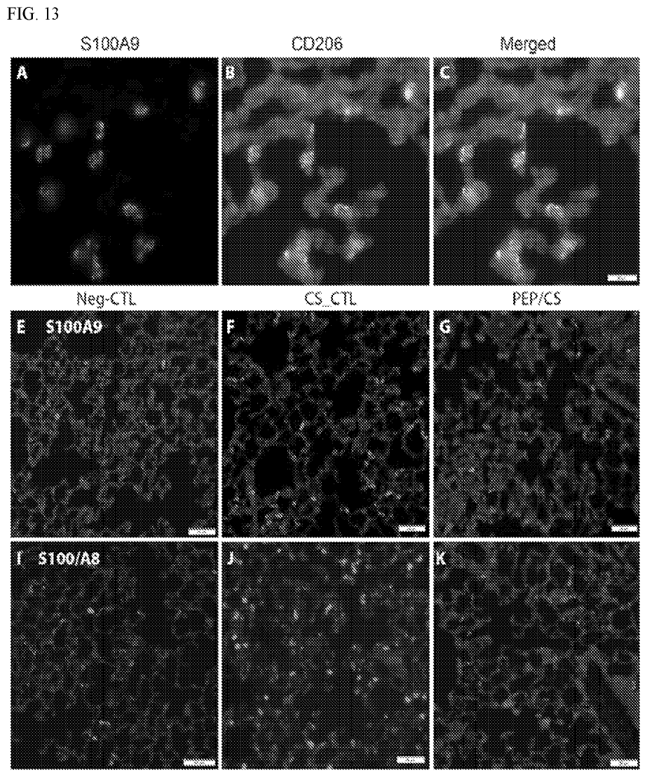

Inflammation is a classical feature of COPD. To determine the effect of PEP nebulization on lung inflammation, a quantitative visual score assessment was performed on representative lung sections (FIG. 8B). Compared to air exposed controls, the cigarette smoke-exposed mice lungs showed a significant increase in lymphocytic infiltrates (FIG. 8B; FIG. 12A) within perivascular regions (P=0.024). The cigarette smoke-exposed mice treated with 15 mg/mL PEP via nebulization showed a more significant increase in lymphocytic infiltration in these regions compared to the cigarette smoke-exposed placebo control mice (p=0.023). A statistically significant increase in lymphocytic infiltration occurred in the nebulized PEP-treated mice exposed to air, (p<0.001; compared to air exposed only control mice) implying that PEP alone causes significant lymphocyte recruitment and retention in the lung. Further immunofluorescence staining of these lymphocytic infiltrates identified an increased subpopulation of CD4⁺FOXP3⁺ double positive T regulatory cells among CD4⁺ T cells in PEP (15 mg/mL) nebulized lungs compared to the cigarette-smoke-exposed placebo control lungs (26±1.3% vs. 17±3%, p=0.05, FIG. 12B-D). Furthermore, immunostaining for B regulatory cell (B-reg) marker CD22 identified an increased CD22-expressing plasma cell subpopulation located in the perivascular and peripheral regions of lymphocytic infiltrates in PEP nebulized lungs compared to the placebo control lungs (FIG. 12E-G). These data suggest that PEP nebulization increases regulatory lymphocytic recruitment in the lung rather than induce inflammation.

To further determine whether PEP nebulization modulates cigarette-smoke-induced lung inflammation, inflammation-related gene expression profiles from whole lung RNA-seq data were re-examined. PEP nebulization at both dosages caused a significant decrease in the transcriptomes of S100A8 and S100A9, members of the S100 family of calcium-binding proteins and damage-associated molecular patterns (DAMPs), compared to the cigarette-smoke-exposed placebo controls. Specifically, for PEP nebulized lungs at 15 mg/mL dosage, a 6.76-fold decrease for S100A8 (p<0.0001) and a 7.06-fold reduction for S100A9 (p<0.0001) was observed compared to the cigarette smoke controls. In contrast, the expression of S100A8/A9 mRNA was significantly increased by cigarette smoke exposure: 2.73-fold for S100A8 and 2.89-fold for S100A9 (p=−0.02 and p=0.01 respectively, compared to sham exposed control).

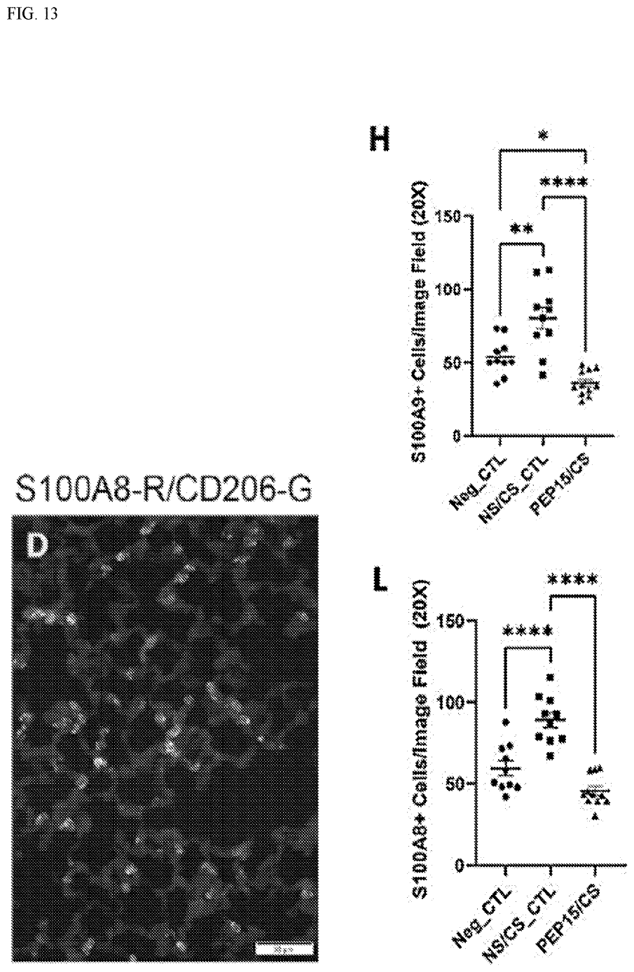

To validate the S100A8/A9 expression at the protein level, immunofluorescence staining was performed on mouse lung tissue sections. Constitutive S100A8/A9 expression was found in the CD206 expressing lung macrophages in the alveolar and interstitial regions across all study groups, including the room air sham control group. However, the number of S100A8/A9 expressing macrophages was significantly increased in the cigarette-smoke-exposed placebo controls from 59±5 (mean±SE) to 89±5 cells/image field (20×) for S100A8 (p<0.001) and 54±4 to 80±7 cells/image field (20×) for S100A9 (p<0.001), compared to the sham controls. Consistent with the RNA-seq result, PEP nebulization at 15 mg/mL dosage markedly reduced this cigarette-smoke-induced increase in S100A8/A9 expressing macrophages from 89±5 to 46±3 cells/image field (20×) for S100A8 (p<0.001) and 80±7 to 36±3 cells/image field (20×) for S100A9 (p<0.001), compared to the cigarette smoke controls (FIG. 13).

### PEP Nebulization Suppresses Cigarette-Smoke-Induced NF-κB Activation and Apoptosis Signaling Pathways by Enhancing Antioxidative Stress Capacity

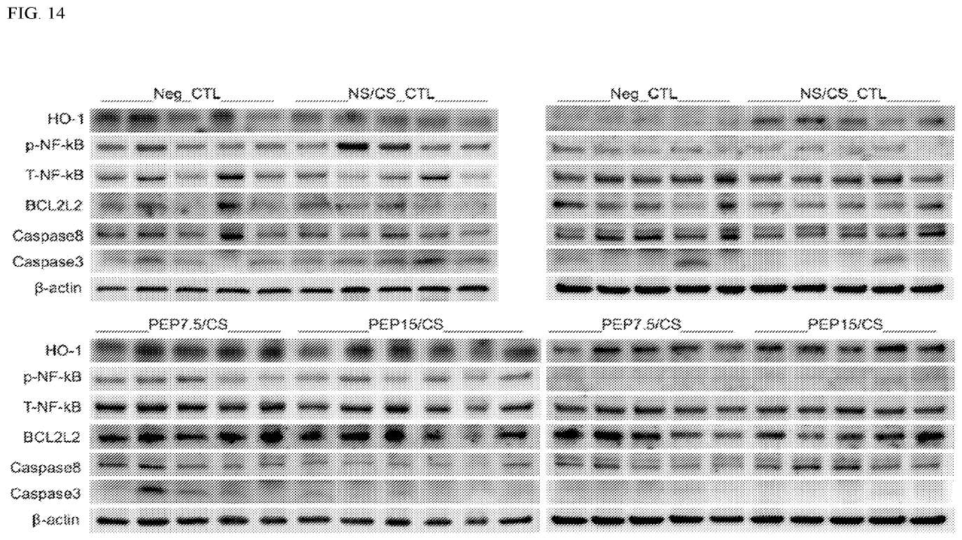

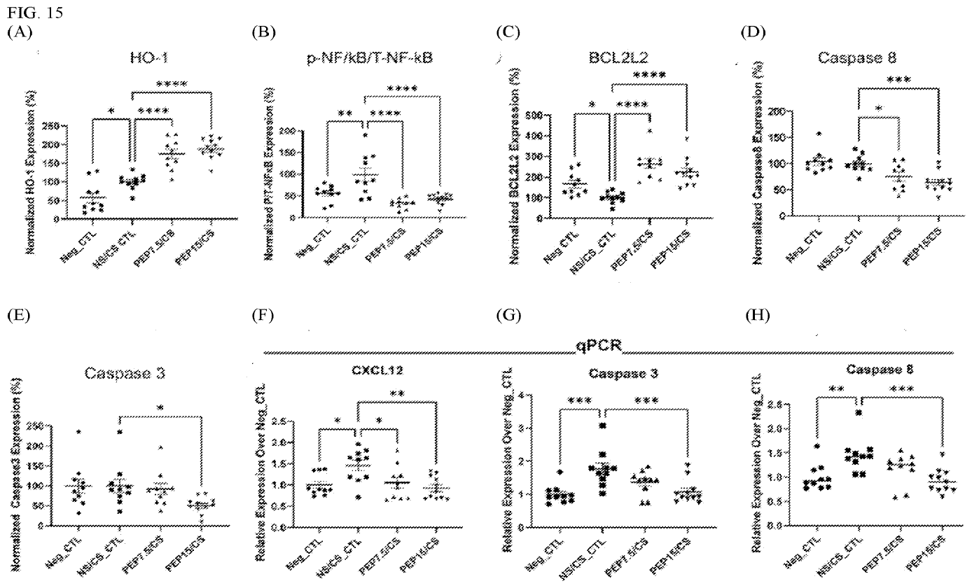

To expand further on the RNA-sequencing results which showed a reduction in inflammation and apoptosis related gene expression, the expression of key pathway molecules was further examined by protein expression. FIG. 14 and FIG. 15A show an increase in the expression of the antioxidant Heme Oxygenase-1 (HO-1) in whole lung lysates from cigarette-smoke-exposed mice (p=0.02 compared with sham controls). Whole lung lysate from mice that received PEP and cigarette smoke exposure showed significantly increased HO-1 protein when compared with the cigarette smoke-exposed mice (p<0.0001, FIG. 15A). This may result from the enrichment of HO-1 and other antioxidant cargo in PEP. Next, the activation of NF-κB and the pro-/anti-apoptosis molecules caspases 3/8, and BCL2L2 were examined. Examination of NF-κB signaling protein P65 revealed activation of NF-κB signal in cigarette-smoke-exposed lungs (p=0.003); levels of p65 were reduced by PEP nebulization at both PEP dosages (p<0.0001) (FIG. 14; FIG. 15B). PEP nebulization also decreased pro-apoptotic caspase 8 in the cigarette smoke-exposed and PEP nebulized lungs (FIG. 14; FIG. 15A-E) in a dose-dependent manner (p=0.03 for low-dose PEP and p=0.0009 for high-dose PEP) and decreased pro-apoptotic caspase 3 (p=0.04) only at the higher concentration (15 mg/mL). High-dose PEP (15 mg/mL) nebulization also reduced caspases 3/8 expression at the transcriptional level (p=0.0009 for Caspase 3 and p=0.0006 for caspase 8; FIG. 15G,H), while these were upregulated in cigarette-smoke-exposed lungs in the absence of PEP treatment (p=0.0004 for caspase 3 and p=0.004 for caspase 8). In addition to influencing apoptosis regulators, PEP nebulization also reduced cigarette smoke-induced proinflammatory chemokine CXCL12 upregulation (p=0.01) at its transcription level in a dose-dependent manner (p=0.02 for low-dose PEP and p=0.002 for high-dose PEP; FIG. 15F).

### PEP Reduces Cigarette Smoke Extract-Induced Apoptosis and Oxidative Stress in Alveolar Epithelial Cells

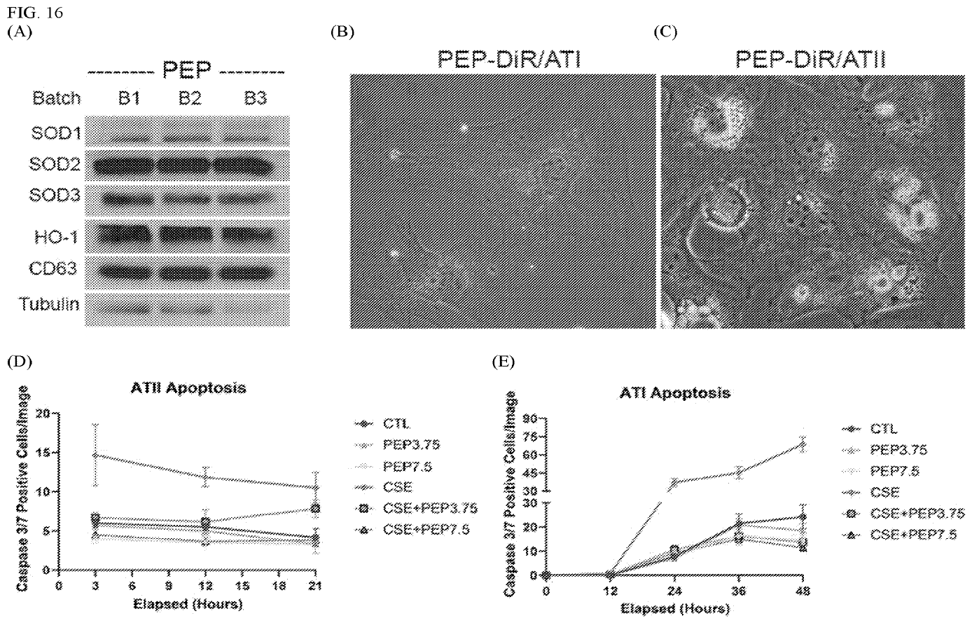

To further elucidate molecular mechanisms by which PEP may reduce oxidative injury in lung cells, primary rat alveolar epithelial type I and type II cells (ATI and ATII) were stimulated with cigarette smoke extract (CSE) in the presence or absence of PEP coincubation. The batch-to-batch variability of PEP (size, concentration, antioxidant activity, and content of protein) is regularly tested, providing an internal control to ensure batch to batch consistency of this exosome product. Western blot analysis from three batches of PEP confirms its enrichment with antioxidants, including SOD 1-3 and heme oxygenase (HO-1) (FIG. 16A). In parallel, to determine whether PEP can be readily taken up by target cells during coincubation, snapshot live cell images were taken immediately after adding DiR-labeled PEP into the cultured ATII and ATI cells. Upon direct contact with the cells, PEP was internalized and localized at the perinuclear regions of the cells (FIG. 16B,C); the uptake peaked 45 minutes after the internalization started.

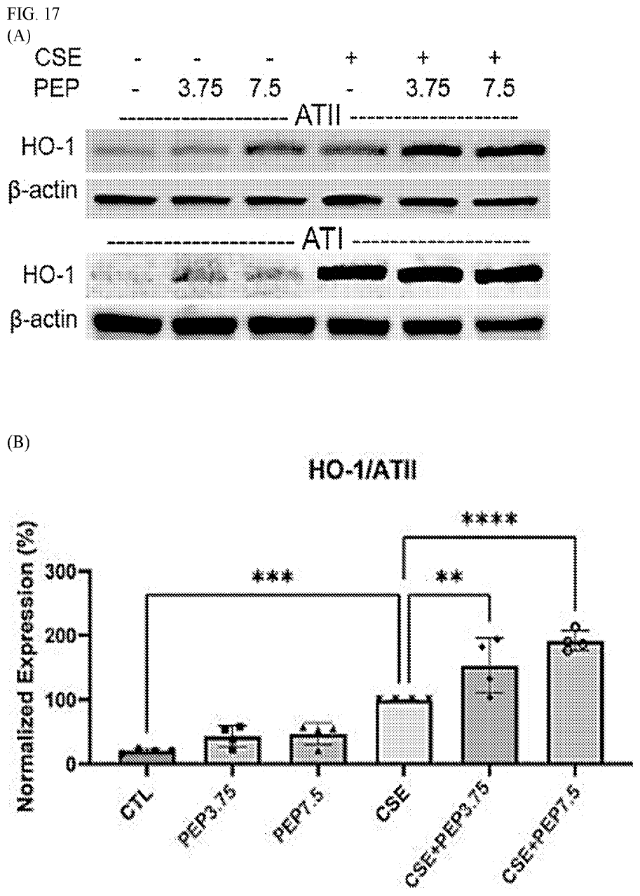

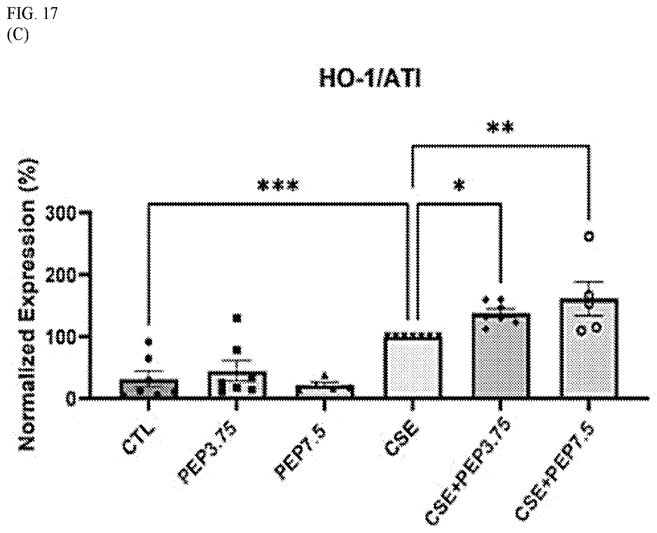

To determine whether PEP reduced apoptosis in cultured alveolar epithelial cells, cigarette-smoke-extract-induced ATII or ATI apoptotic cells were fluorescently labeled with caspases 3/7 (FIG. 16D,E) and imaged. In ATII cells, due to their propensity to transdifferentiate in cell culture, the apoptosis assay was conducted within 21 hours of adding cigarette smoke extract on day 2 of culture. An average of 10-15 apoptotic cells per image field were identified in CSE-stimulated control cells. In comparison, cells preincubated with PEP at all concentrations (24 hours) showed significantly fewer apoptotic cells (7.5 cells/image field) after stimulation with cigarette smoke extract (p<0.001). PEP controls without cigarette smoke extract stimulation showed similarly fewer apoptotic cells as the normal controls without cigarette smoke extract stimulation (FIG. 16D). In ATI cells, caspase 3/7 labeled cells increased over time (0-48 hours) from 0 cells/image field to 75 cells/image field in CSE-stimulated cells. In contrast, the number of apoptotic cells was significantly reduced in PEP preincubated cells after cigarette smoke extract stimulation and was similar to the normal controls cultured in the absence of cigarette smoke extract (FIG. 16E, p<0.001). Next, to determine whether PEP enhances cellular antioxidative stress capacity in the recipient cells, western blot analysis was performed on cultured ATII and ATI cells with or without coincubation of PEP and cigarette smoke extract coincubation. The level of cellular HO-1 protein was increased in CSE-stimulated ATII (p<0.001) and ATI (p<0.001) cells relative to unstimulated cells. PEP preincubation further increased HO-1 expression in both ATII (p=0.008 for 3.75 mg/mL PEP and p<0.001 for 7.5 mg/mL PEP) and ATI (p=0.04 for 3.75 mg/mL PEP and p=0.004 for 7.5 mg/mL PEP) cells in a dose-dependent manner (FIG. 17). This increase in cellular HO-1 protein is consistent with the observed increase in HO-1 protein in murine lungs exposed to both cigarette smoke and nebulized PEP.

### Pulmonary Exosome Delivery Procedures

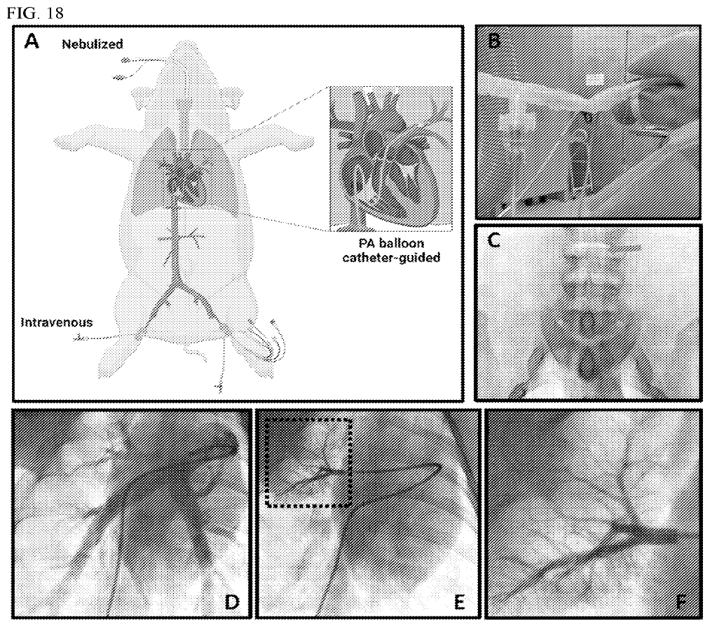

To achieve targeted pulmonary delivery of PEP, the following delivery methods were tested: nebulized, PA balloon catheter-guided, and intravenous. A cartoon graphic demonstrating these approaches is shown in FIG. 18A. Nebulization was accomplished with a jet nebulizer attached to an endotracheal tube (FIG. 18B). A radiographic image demonstrating endotracheal tube position above carina is shown (FIG. 18C). To facilitate the PA balloon catheter-guided delivery, pulmonary angiograms were acquired (FIG. 18D). The balloon catheter was then used to selectively enter a single branch of the pulmonary artery. The balloon was wedged to occlude forward blood flow, shown with contrast in FIG. 18E-18F. Blood flow occlusion with PEP delivery in the pulmonary artery branch for five minutes allowed for exosome absorption. Intravenous PEP delivery was performed through the femoral vein.

### Biodistribution of PEP in Lung Tissue

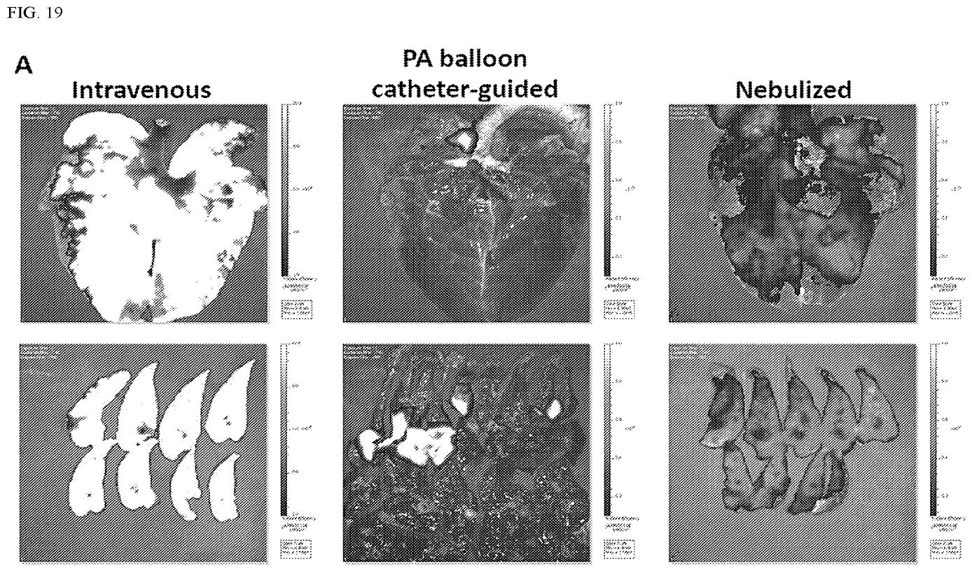

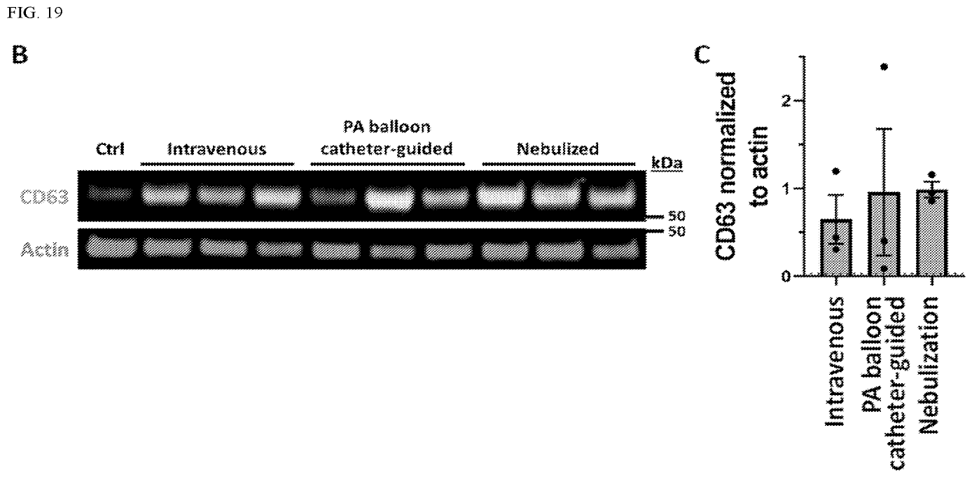

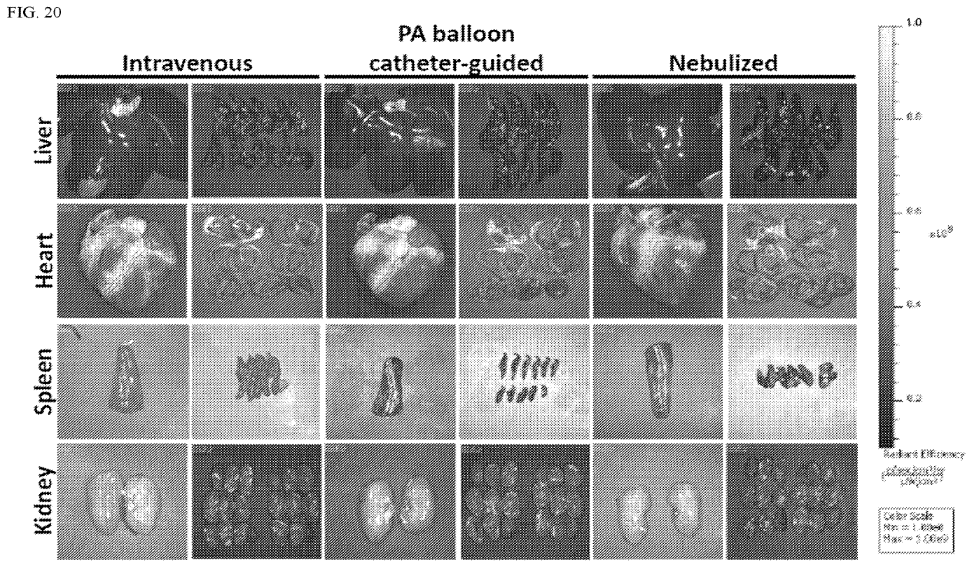

Following PEP delivery by intravenous, PA balloon catheter-guided, or nebulized approaches, lung tissue was removed and imaged with Xenogen IVUS to track far-red DiR dye. Intravenous and nebulized methods achieved global lung delivery, while the PA balloon catheter-guided approach was able to localize delivery to a single area (FIG. 19A). Western blot was used to demonstrate presence of exosomal protein CD63 in lung tissue (FIG. 19B). Quantification of CD63 normalized to actin demonstrated that nebulization had the highest delivery with mean of 0.989 (+/−SD 0.15) compared to level of control at 0.029 (FIG. 19C). PA balloon catheter resulted in a similarly high mean of 0.959 (+/−1.24 SD) with greater variation between animals (FIG. 19C). Intravenous delivery resulted in the lowest mean at 0.649 (+/−0.47 SD)(FIG. 19C).

### Biodistribution of PEP in Off-Target Tissues

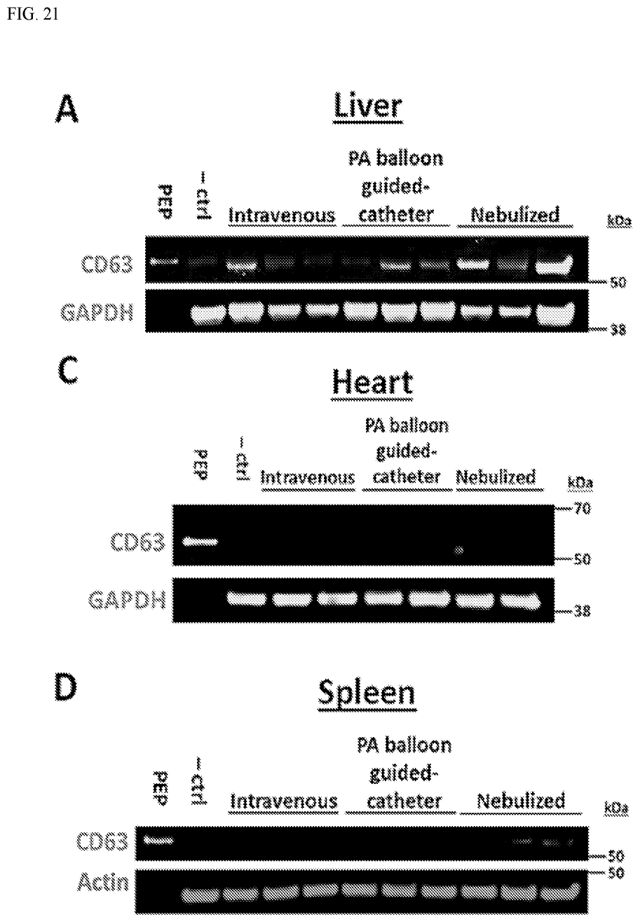

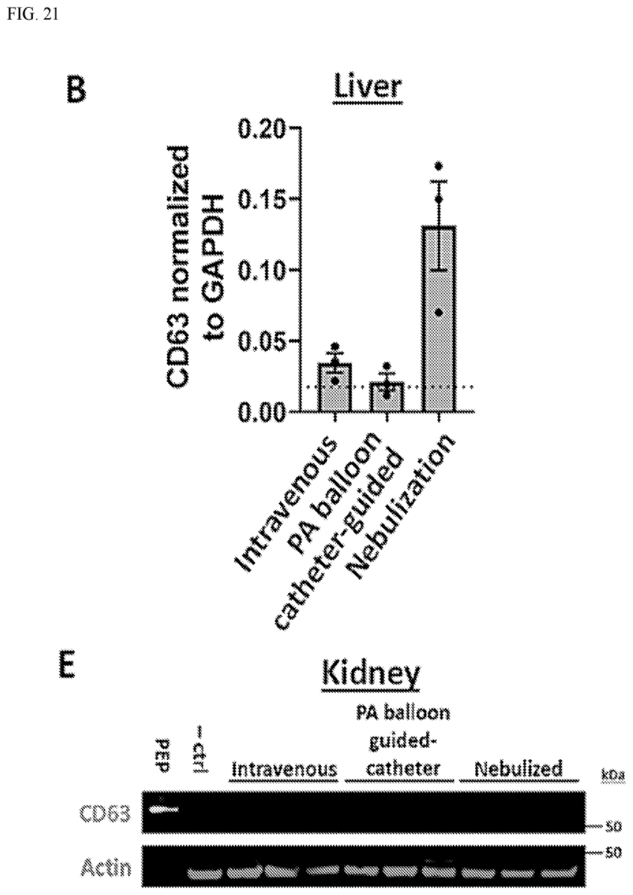

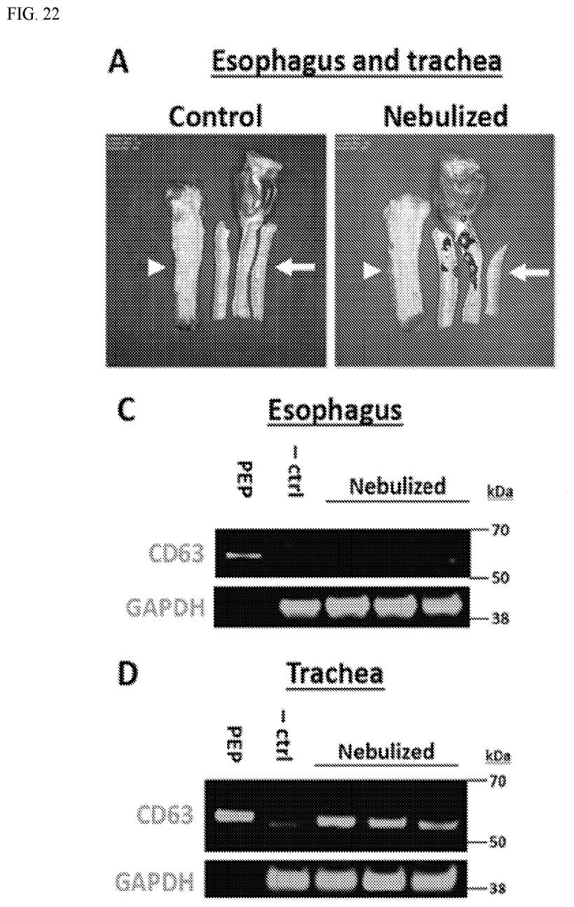

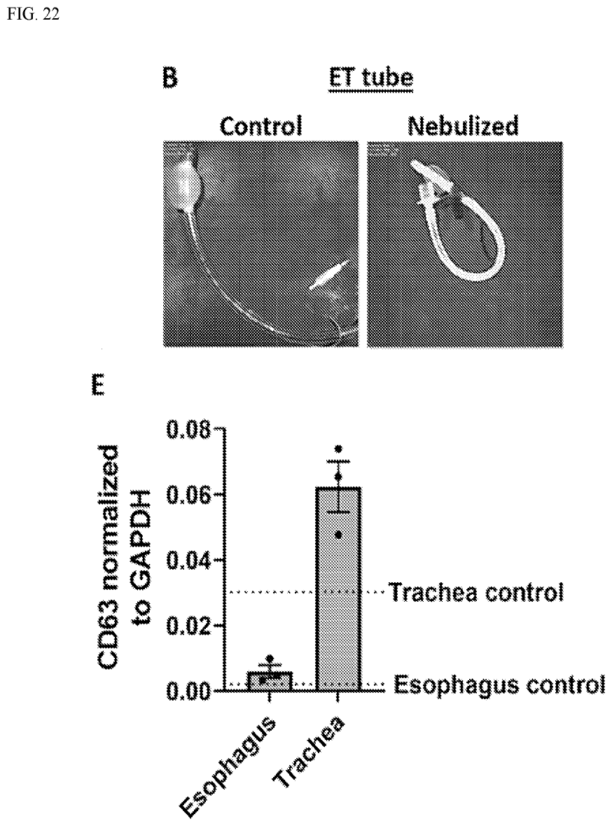

PEP uptake in off-target organs including heart, liver, spleen, and kidney was similarly evaluated by Xenogen IUS imaging. No uptake was noted in the liver, heart, spleen, or kidney with any of the methods (FIG. 20). To assess PEP tissue uptake at higher resolution, Western blot of CD63 was utilized. Liver tissue showed variable presence of PEP between different methods of administration (FIG. 21A). Nebulized animals showed the greatest amount of liver uptake with quantification of CD63 fluorescent signal normalized to GAPDH of 0.131 (+/−0.05 SD) compared to control liver tissue at 0.017, while intravenous (mean 0.034+/−0.01 SD) and PA balloon catheter-guided (mean 0.021+/−0.01 SD) had levels that were closer to control tissue (FIG. 21B). CD63 protein was not detected in the heart, spleen, or kidney samples of PEP-treated animals. For nebulized animals, the esophagus and trachea were also evaluated (FIG. 22A-B). Xenogen IVUS imaging demonstrated no esophageal signal, but scattered signal in the trachea (FIG. 22A). The endotracheal tube was also evaluated after nebulization in comparison to an endotracheal tube without exposure to nebulized PEP. After nebulization, there was PEP signal throughout the entire endotracheal tube (FIG. 228). Western blot confirmed absence of PEP in esophagus (FIG. 22C) and presence of PEP in trachea (FIG. 22D). Quantification demonstrated mean CD63 signal normalized to GAPDH in the esophagus of nebulized animals (0.005-1-0.003 SD) was similar to control esophagus (mean 0.002), while the trachea showed greater CD63 (mean 0.062+/−0.01 SD) compared to control trachea at 0.030 (FIG. 22E).

### Histologic Analysis of PEP in Lung Tissue

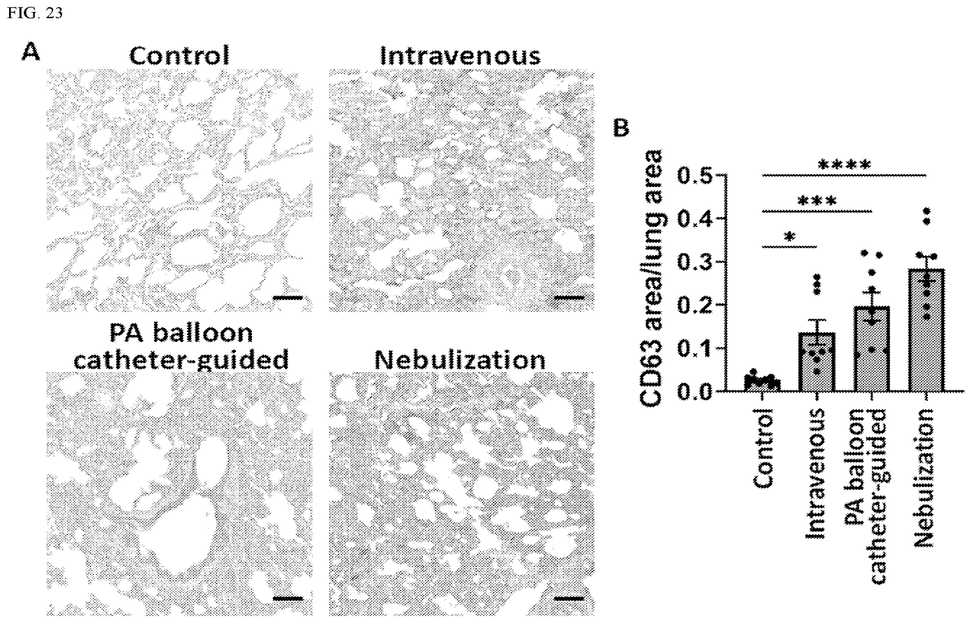

To further demonstrate PEP presence in lung tissue and demonstrate the specific areas of tissue uptake, immunohistochemical staining was performed for CD63. Compared to control lung tissue which showed minimal staining, there was CD63 detected in animals treated by intravenous, PA balloon catheter-guided, and nebulization approaches (FIG. 23A). Quantification demonstrated significant increase in CD63 detected per total tissue area in nebulization (mean 0.283+/−0.02 SEM, p=0.0344), PA balloon catheter-guided (mean 0.196 +/−0.03 SEM, p=0.0009), and intravenous delivery (mean 0.137+/−0.02 SEM, p<0.0001) compared to control lung (mean 0.026+/−0.01 SEM)(FIG. 23B). These results recapitulate the trend seen by western blot detection with the greatest CD63 signal detected in nebulization followed by PA balloon catheter-guided and lastly intravenous delivery.

This disclosure therefore describes the efficacy of PEP as a novel approach to treat cigarette-smoke-induced emphysema using a relevant mouse model. Nebulized PEP effectively delivers antioxidant and immunomodulatory molecules into the alveolar regions of murine lungs and can attenuate cigarette-smoke-induced oxidative stress, inflammation, and/or apoptosis in the lung. PEP suppresses cigarette-smoke-induced emphysema by altering relevant disease inducing pathways activated by cigarette smoke in the lung. Immuno-oncology gene panel RNA sequencing and lung tissue western blot analysis revealed that PEP nebulization reduced cigarette smoke-induced oxidative stress-initiated, NF-kB-mediated apoptotic cell death in the lungs. Immunohistostaining of lung tissue sections also suggest that nebulized PEP has an immunomodulatory role by increasing immune regulatory lymphocytes and decreasing macrophage-mediated inflammatory processes. Parallel in vitro studies showed that the incubating alveolar epithelial cells with PEP prior to an oxidative challenge with cigarette smoke extract reduces oxidative injury to the cells and apoptotic cell death. These results demonstrate that nebulized PEP enables direct delivery of exosome cargo to alveolar epithelial cells and macrophages. The delivery of PEP by nebulization provides antioxidative activity and immunomodulation, leading to suppression of cigarette-smoke-induced inflammatory response and apoptotic cell death and subsequent attenuation of emphysema in marine lungs.

Using RNA sequencing and immunoblotting techniques, several molecular mechanisms were identified by which PEP counteracts cigarette smoke injury. The RNA-seq data revealed that CSE-induced upregulation of apoptosis signaling pathways in the emphysematous lungs was at least partially inhibited by PEP. PEP also reduction caspases 3/8 and the BCL2 family proteins in whole lungs in the group that received nebulized PEP. In addition, the observed suppression of ATII and ATI in vitro apoptosis by cigarette smoke extract provides further support for the efficacy of PEP in treating cigarette smoke-induced apoptotic death in alveolar epithelial cells. The suppression of apoptotic cell death by PEP is important as it provides a molecular rationale for the use of nebulized PEP to prevent alveolar cell loss in COPD.

Inflammation and immune cell infiltration is a characteristic feature of human COPD and was also evident in the cigarette-smoke-induced emphysema model. Significantly increased S100A8/A9-producing macrophages and lymphocytic infiltrates were observed in cigarette-smoke-exposed placebo control lungs. However, the extent of this lymphocytic infiltration was even more prominent in PEP-treated lungs. Deeper phenotyping revealed that the increased lymphocytic infiltration in PEP nebulized lungs is accompanied by an increased proportion of both CD4⁺FOXP3⁺ Tregs and CD22⁺ plasma cells, indicating an expansion of regulatory T cells and B cell resulting from treatment with PEP. Evidence in support of an immunoregulatory role is also provided by the IPA analysis of RNA-seq data, in which both Th1 and Th2 signaling pathways were enhanced in PEP-treated lungs. Decreased Treg activity has been linked to a higher proportion of proinflammatory B lymphocytes and T lymphocytes in the BAL samples from COPD patients, and decreased Treg counts and POXP3 expression have been correlated with COPD severity.

In addition to the effects on T lymphocytes and B lymphocytes, a significantly reduced recruitment of S100A8/A9-producing macrophages was also seen in PEP-treated lungs. S100A8/A9 are members of the S100 family of calcium-binding proteins and damage-associated molecular patterns (DAMPs). These receptors are constitutively expressed and anti-inflammatory in a healthy state but transition into pro-inflammatory signals under oxidative stress-associated pathological conditions, which results in their release into the extracellular space via activated macrophages. Patients with COPD often have elevated S100A8 and S100A9 in plasma and bronchoalveolar lavage fluid, and exposure to cigarette smoke increases S100A9 levels in age-matched mice. In an animal model, depletion of S100A9 or loss of S100A9 signaling attenuated cigarette-smoke-induced airspace enlargement, alveolar destruction, and MMP-3, MMP-9, MCP-1, IL6, and Cxcl1 release. Alveolar macrophages are integral to COPD development and are functionally equipped to be both the initiator and perpetuator of lung injury in response to cigarette smoke. Together, the reduction in inflammatory macrophage activation and increased Tregs and plasma cells by PEP support an immunoregulatory role for PEP in treating COPD.

Cigarette smoking is the most common cause of COPD. Excessive generation of reactive oxygen species (ROS) during chronic exposure to cigarette smoke results in dysregulation of redox homeostasis, which leads to oxidative stress and oxidative cellular injury. Antioxidant enzyme upregulation is one of the cellular defense mechanisms against oxidative stress to maintain redox homeostasis. The current study suggests that antioxidant capacity augmented by treatment with antioxidant-enriched PEP exosomes can bolster this cellular defense mechanism. Western blot analysis revealed upregulated antioxidant HO-1 in the lungs exposed to cigarette smoke. PEP treatment replenished HO-1 protein in the lungs. This was further supported by the result from ATII and ATIs in vitro cell culture.

Oxidative stress and apoptosis are central to the pathogenesis of COPD and represent interconnected biological processes. The transcription factor nuclear factor-KB (NF-κB) regulates oxidative stress and apoptosis in many cell types. Reactive oxygen species (ROS) burden generated from oxidative stress inducers like cigarette smoke can cause phosphorylation and degradation of inhibitor of nuclear factor kappa B (1κB), leading to NF-κB dimer (e.g., p65/p50 subunits) nuclear translocation and activation of the NF-κB signaling pathway. NF-κB activation promotes apoptosis in response to cellular stress, in part by suppressing anti-apoptotic and activating pro-apoptotic gene expression. NF-κB p65 was activated in the cigarette-smoke-exposed emphysematous lungs, while NF-κB p65 activation was significantly inhibited in the cigarette-smoke-exposed lungs after treatment with PEP. Furthermore, following this NF-κB activation, pro-apoptotic genes caspase 3 and caspase 8 were upregulated in cigarette-smoke-exposed control lungs, but downregulated in cigarette-smoke-exposed lungs treated with PEP. Finally, the anti-apoptosis molecule BCL2L2 was downregulated in cigarette-smoke-exposed control lungs but was upregulated after treatment with PEP.

In summary, the cigarette-smoke-induced murine emphysema model accurately reflects COPD pathogenesis. The balance between oxidative stress and antioxidant activity is disrupted. The balance between oxidative stress-initiated cell apoptosis and regeneration also is disrupted, which leads to inflammation and destruction of alveolar units. As a therapeutic agent, PEP targets both sides of this balance by suppressing apoptosis and inflammatory signaling and/or promoting anti-inflammatory immune response as well as wound healing and repair simultaneously.

Thus, this disclosure describes compositions and methods for treating a pulmonary condition in a subject. The method generally includes administering an effective amount of a PEP composition to a subject.

As used herein, “treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Treatment that is prophylactic—e.g., initiated before a subject manifests a symptom or clinical sign of the condition such as, for example, while tissue damage remains subclinical—is referred to herein as treatment of a subject that is “at risk” of having the condition. As used herein, the term “at risk” refers to a subject that may or may not actually possess the described risk. Thus, for example, a subject “at risk” of a pulmonary condition is a subject possessing one or more risk factors associated with the pulmonary condition such as, for example, genetic predisposition, ancestry, age, sex, geographical location, lifestyle, occupation, exposure (whether actual or anticipated) to airborne toxins, or medical history. Treatment may also be continued after symptoms have resolved, for example to prevent or delay their recurrence.

Accordingly, a PEP composition can be administered before, during, or after the subject first exhibits a symptom or clinical sign of the pulmonary condition. Treatment initiated before the subject first exhibits a symptom or clinical sign associated with the pulmonary condition may result in decreasing the likelihood that the subject experiences clinical evidence of the pulmonary condition compared to a subject to which the PEP composition is not administered, decreasing the severity of symptoms and/or clinical signs of the pulmonary condition compared to a subject to which the PEP composition is not administered, and/or completely resolving the pulmonary condition. Treatment initiated after the subject first exhibits a symptom or clinical sign associated with the pulmonary condition may result in decreasing the severity of symptoms and/or clinical signs of the pulmonary condition compared to a subject to which the PEP composition is not administered, and/or completely resolving the condition.

Thus, the method includes administering an effective amount of the PEP composition to a subject having, or at risk of having, a particular condition. In this aspect, an “effective amount” is an amount effective to reduce, limit progression, ameliorate, or resolve, to any extent, a symptom or clinical sign related to the condition. In this regard, an “effective amount” is an amount effective to decrease lung compliance (Cst), decrease mean linear intercept (Lm), increased lymphocytic infiltrates in perivascular areas, increased proportion of CD4⁺FOXP3⁺ Tregs in perivascular lymphocytes, increased proportion of CD22⁺ plasma cells in perivascular lymphocytes, decreased expression of inflammatory signaling pathway genes, decreased expression of fibrotic signaling genes, decreased expression of PD1/PD-L1 cancer immunotherapy genes, decreased expression of tumor microenvironment pathway genes, decreased phagosome formation, decreased expression of tumor necrosis factor (TNF), decreased expression of IL-17A-regulated pathways, decreased immune cell chemotaxis, decreased expression of NF-κB signaling pathway genes, decreased expression of senescence pathway genes, increased expression of wound healing pathway genes, decreased S100A8⁺ macrophages in alveolar and/or interstitial regions of the lung, decreased S100A9⁺ macrophages in alveolar and/or interstitial regions of the lung, increased Heme Oxygenase-1 (HO-1) in awhole lung lysate, decreased NF-κB p65 in a whole lung lysate, decreased caspase 8 in a whole lung lysate, decreased caspase 3 in a whole lung lysate, decreased CXCL12 transcription, increased expression of antioxidants by alveolar epithelial cells (e.g., SOD 1-3 and heme oxygenase (HO-1)), decreased apoptosis of alveolar epithelial cells, increased lung capacity, increased lung function, and increased quality of life or any combination of two or more of the foregoing, compared to a subject to which the PEP composition is not administered.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

While described herein in the context of treating COPD, the compositions and methods described herein may involve treating any pulmonary condition. Exemplary alternative pulmonary conditions treatable using a PEP composition as described herein include, but are not limited to, alveolar epithelial cell injury, alveolar inflammation, macrophage-mediated lung injury, chronic obstructive pulmonary disease (COPD), emphysema, interstitial lung diseases (ILD), pulmonary fibrosis, acute respiratory distress syndrome (ARDS), acute lung injury (ALI), or toxic agent exposure.

A PEP composition may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, hydrogel, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue, tissue adhesive, or a supportive matrix (e.g., a collagen scaffold).

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intradermal, transcutaneous, rectally, etc.). A pharmaceutical composition can be administered to a mucosal surface, such as by administration to, for example, the nasal or respiratory mucosa (e.g., by inhalation or nebulization). A pharmaceutical composition also can be administered via a sustained or delayed release.

Thus, a pharmaceutical composition may be provided in any suitable form including but not limited to a solution, a suspension, an emulsion, a spray, an aerosol, a nebulized formulation, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both.

In one or more embodiments, the composition may be nebulized. This disclosure demonstrates the benefit of delivering inflammation-targeted and/or apoptosis-targeted therapeutics to distal alveolar areas of the lung to reduce injury due to pulmonary disease. Thus in one or more embodiments, nebulized delivery of PEP exosomes to distal alveolar epithelial cells may be preferred. This novel approach to pharmacotherapy delivery specifically targets alveolar cells with high drug concentrations, which may allow treatment using a smaller dose of PEP than if the PEP composition is administered systemically. When certain therapeutics for treating pulmonary conditions are administered systemically, they must be administered at near-toxic doses in order to have an effective dose reach the lungs. In contrast, nebulized PEP delivered directly to the pulmonary tract (e.g., direct targeting to the lung bed) reduces or eliminates the extent to which near-toxic systemic doses of conventional therapeutics would need to be required.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from about 1×10⁶ PEP exosomes to about 1×10¹⁵ PEP exosomes to the subject, although in one or more embodiments the methods may be performed by administering PEP in a dose outside this range.

In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10⁻¹⁰ PEP exosomes, at least 1.25×10¹⁰ PEP exosomes, at least 2.5×10¹⁰ PEP exosomes, at least 5×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least Ix10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1 ×10¹⁴ PEP exosomes, no more than 1×10¹³ PEP exosomes, no more than 1×10¹² PEP exosomes, no more than 1×10¹¹ PEP exosomes, no more than 5×10¹⁰ PEP exosomes, or no more than 1×10¹⁰ PEP exosomes.

In one or more embodiments, the method can include administering sufficient PEP to provide a dose characterized as a range having endpoints defined by any a minimum dose identified above and any maximum dose identified above that is greater than the selected minimum dose. For example, in one or more embodiments, the method can include administering sufficient PEP to provide a dose of from 1×10¹⁰ to 1×10¹³ PEP exosomes such as, for example, a dose of from 1×10¹¹ to 5×10¹² PEP exosomes, a dose of from 1×10¹⁰ to 1×10¹¹ PEP exosomes, a dose of from 2.5×10¹⁰ to 5×10¹⁰ PEP exosomes, or a dose of from 1.25×10¹⁰ to 5×10¹⁰ PEP exosomes.

In one or more embodiments, the method can include administering sufficient PEP to provide a dose that is equal to any minimum dose or any maximum dose listed above. Thus, for example, the method can involve administering a dose of 1×10¹⁰ PEP exosomes, 1.25×10¹⁰ PEP exosomes, 2.5×10¹⁰ PEP exosomes, 5×10¹⁰ PEP exosomes, 1×10¹¹ PEP exosomes, 1×10¹² PEP exosomes, 5×10¹² PEP exosomes, 1×10¹³ PEP exosomes, or 1×10¹⁴ PEP exosomes.

Alternatively, a dose of PEP can be measured in terms of the concentration of PEP upon reconstitution from a lyophilized state. Thus, in one or more embodiments, the methods can include administering PEP to a subject at a dose of, for example, from about a 0.01% solution to a 100% solution to the subject, although in one or more embodiments the methods may be performed by administering PEP in a dose outside this range. As used herein, a 100% solution of PEP refers to one vial of PEP (approximately 2×10¹¹ exosomes or 75 mg) solubilized in i mL of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose of may be administered as a single dose, continuously over 24 hours, as two administrations, which may be equal or unequal. When multiple administrations are used to deliver a single dose, the interval between administrations may be the same or different. In certain embodiments, PEP may be administered as a once-off administration, for example, during a surgical procedure.

In certain embodiments in which multiple administrations of the PEP composition are administered to the subject, the PEP composition may be administered as needed to treat the pulmonary condition to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP to a subject at an interval (for two administrations) or intervals (for more than two administrations) characterized by a range having endpoints defined by any minimum interval identified above and any maximum interval that is greater than the minimum interval. For example, in one or more embodiments, the method can include multiple administrations of PEP at an interval or intervals of from one day to six months such as, for example, from three days to ten days. In certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique cargo profile—e.g., protein composition and/or gene expression. In this way, the PEP composition can provide a broader spectrum of therapeutic activity than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an.” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the word “exemplary” is used interchangeably with “illustrative” and should not be construed to indicate that an embodiment is preferred or advantageous over other embodiments.

For any method disclosed herein that includes discrete steps, the steps may be performed in any feasible order. And, as appropriate, any combination of two or more steps may be performed simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXAMPLES

The present invention is illustrated by the following examples. It is to be understood that the particular examples, materials, amounts, and procedures are to be interpreted broadly in accordance with the scope and spirit of the invention as set forth herein.

### Cells and Reagents

Primary rat ATII and ATI cells were isolated from female Sprague Dawley rats following a well-established protocol previously described (Wang, S., and Hubmayr, R. D. (2011) Am J Respir Cell Mol Biol 44:692-699; Weller, N. K., and Karnovsky, M. J. (1986) Am. Pathol 122:92-100; Gonzalez, R. F., and Dobbs, L. G. (2013) Methods Mol Biol 945:145-159). Freshly isolated cells were cultured in high-glucose Dulbecco's Modified Eagles Medium (DMEM) supplemented with 10% FBS, 200 units (μg)/mL of penicillin, and streptomycin, maintained in a 5% CO₂ humidified 37° C. incubator. Day 2-3 of ATII culture and Day 5-7 ATI culture were used for all the experiments carried out in this study.

Primary antibodies used included mouse anti-human-specific CD63 (abcam, Cambridge, United Kingdom; catalog # ab271286), mouse anti-SOD2 (R&D Systems, Minneapolis, MN; catalog #MAB3419), rabbit anti-HO-1 (MilliporeSigma, Burlington, MA; catalog #374090-100UL), SOD1,2,3, mouse anti-caspase 3 (Proteintech Group, Inc., Rosemont, IL; catalog #66470-1-Ig), rabbit anti-caspase 8 (Proteintech Group, Inc., Rosemont, IL; catalog #13423-1-AP), rabbit anti Phospho-NF-κB p65 (Ser536) (93H1)(Cell Signaling Technology, Inc., Danvers, MA; catiog #3033S), Rabbit anti NF-κB p65 (D14E12) (Cell Signaling Technology. Inc., Danvers, MA; catalog #f 8242S), rabbit anti-β-actin (Cell Signaling Technology, Inc., Danvers, MA; catalog #4967s), rabbit anti-S100A8 (Proteintech Group, Inc., Rosemont, IL; catalog #15792-1-AP), rabbit anti-S100A9 (Proteintech Group, Inc., Rosemont, IL; catalog #26992-1-AP), mouse anti-CD206 (Proteintech Group, Inc., Rosemont, IL; catalog #60143-1-Ig), mouse anti-CD22 (ThermoFisher Scientific, Inc., Waltham, MA; catalog #TA506412S), rat anti-CD20 (abeam, Cambridge, United Kingdom; catalog #AB271288), rabbit anti-CD4 (abcam, Cambridge, United Kingdom; catalog #AB288724), rat anti-FOXP3 (ThermoFisher Scientific, Inc., Waltham, MA; catalog #14-5773-80).

For Western blot using an ODYSSEY Fe imaging system (LI-COR Biosciences, Inc., Lincoln, NE), secondary antibodies are Alexa Fluor 680 goat anti-rabbit (Jackson Immuno Research Laboratories Inc., West Grove, PA; catalog #111-625-144) and Alexa Fluor790 goat anti-mouse (Jackson Immuno Research Laboratories Inc., West Grove, PA; catalog #115-655-146). For immunofluorescence staining, secondary antibodies are Alexa Fluor 488 goat anti-mouse IgG (H+L)(ThermoFisher Scientific, Inc., Waltham, MA; catalog #A32731), Alexa Fluor 568 goat anti-mouse IgG (H+L) (ThermoFisher Scientific, Inc., Waltham, MA; catalog #A-11004), Alexa Fluor 488 goat anti-rat IgG (H+L) (ThermoFisher Scientific, Inc., Waltham, MA; catalog #A-11006), and Alexa Fluor 555 goat anti-rabbit IgG (H+L) (ThermoFisher Scientific. Inc., Waltham, MA; catalog #A-21428).

### PEP Preparation

PEP was produced at Advanced Product Incubator (API, Rochester, MN), a cGMP manufacturing facility supported by the Mayo Clinic (Rochester, MN) to bring translational products into clinical trials. Briefly, pooled human donor platelets were subjected to freeze-thaw, filtration, and lyophilization cycles according as described in US Patent Publication No. 2016/0324794 A1 and U.S. Pat. No. 10,596,123. The resulting product was a stabilized lyophilized powder stored at room temperature and reconstituted before use. Each batch was rigorously tested for microbial contamination, particle size, particle number, and biological activity. PEP batch to batch viability assessment demonstrated stable batch production for the size and number of PEP generated each batch as well as the antioxidant enzymes PEP contained. Rion, LLC (Rochester, MN) provided all PEP used in this study.

For in vivo animal experiments, PEP was reconstituted in distilled water at two concentrations: 7.5 mg/mL (approximately 2.5×10¹⁰ EVs/mL) and 15 mg/mL (approximately 5×10¹⁰ EVs/mL) of PEP particles (reconstituted using 10 mL or 5 mL of H₂O from one vial of lyophilized PEP For in vitro experiments, PEP was reconstituted in a calcium-free DMEM medium at two concentrations: 3.75 mg/mL (approximately 1.25×10¹⁰ EVs/mL) or 7.5 mg/mL (approximately 2.5×10¹⁰ EVs/ml). All PEP preparations were filtered thrugh a 0.45 μm filter after being reconstituted.

To optimize the route of PEP delivery into distal alveolar epithelial cells, PEP was fluorescently labeled with carbocyanine DiOC18 (DiR) dye (ThermoFisher Scientific, Inc., Waltham, MA; catalog #D12731) after being reconstituted as described above for in vivo experiments and filtrated through a 0.2 μm filter. DiR-labeled PEP at two different doses (2.5×10⁹ EVs/mL and 5×10⁹ EVs/mL) was nebulized into spontaneously breathing mice that were constrained in a mouse constrainer using a murine nebulization system (SCIREQ Scientific Respiratory Equipment, Inc., Montreal, Canada) five minutes a day for 5, 10, 15, 20, or 25 days. The biodistribution of DiR-labeled PEP in each lobe of the lungs and other internal organs, including the heart, trachea, stomach, liver, kidneys, intestines, and colon, was examined in using an in vivo imaging system (Xenogen IVIS Spectrum, PerkinElmer, Inc., Waltham, MA).

### Cigarette Smoke Induced Murine Emphysema Model

8-12 months old C57BL6 mice were exposed to cigarette smoke generated by 1R6F Kentucky research cigarettes (Vassallo, et al., (2014) Clinical Immunology 152:25-35) in an automated Boxco Smoke Generator, a state-of-art smoke generation system manufactured by Data Sciences International (DSI). In this system, mice were exposed to a mixture of mainstream and side-stream cigarette smoke in a mass dosing chamber for three hours per day, five days per week for four months to induce pulmonary emphysema. This enables exposure of mice to the levels of cigarette smoke inhalation analogous to one pack of cigarettes a day smoker. This smoke generator has been successfully used to study carcinogenic and other effects of tobacco smoke (Churg et al., 2008, Am J Physiol Lung Cell Mol Physiol 294:L612-631; Fricker et al., 2014, Exper Opin Drug Discov 9:629-64522).

PEP Nebulization into Spontaneous Breathing Mice

PEP was nebulized for delivery to murine lungs for the therapeutic purpose of using PEP to treat cigarette smoke-induced emphysema. Nebulization was achieved in an inhalation tower. This device is a state-of-art nose-only inhalation exposure system (BUXCO, Data Sciences International, Inc., St. Paul, MN), which allows for adjustable pressure, temperature, and humidity control of the PEP delivered. In addition, this system enables simultaneous monitoring of accumulated inhaled PEP and lung function parameters of the mice during PEP nebulization. Mice were constrained in mouse constrainers, which were tightly connected to the inhalation tower, with only their noses exposed to the PEP aerosol generated from the tower. Aerosolized PEP at concentrations of 7.5 mg/mL (approximately 2.5×10¹⁰ EVs/mL) or 15 mg/mL (approximately 5×10¹⁰ EVs/mL) was nebulized into mice to target accumulated inhaled aerosol (AIA) of 0.7 mg in approximately 30 minutes every Monday, Wednesday, and Friday for four weeks. Placebo control mice were exposed to an aerosol generated from normal saline at a similar duration and frequency match PEP nebulization.

### Evaluation of Lung Mechanics

Deeply anesthetized mice who underwent tracheotomy were connected to a flexiVent (SCIREQ Scientific Respiratory Equipment, Inc., Montreal, Canada) for ventilation and lung mechanics measurement as described previously (Lin et al., (2021) Clin Immunol 230:108813). Following anesthesia, the paralytic drug rocuronium was administered to the mice. Lung mechanics were measured and lung compliance/resistance was determined. The static lung compliance (Cst) and pressure-volume curves (PV curves) were computed later to reflect lung function. After mice were euthanized, lung and other internal organs were harvested. Four right lung lobes were immediately frozen for gene and protein expression analysis. The entire left lung was instilled with 1 mL of 4% paraformaldehyde at a standard inflating pressure of 25 cm H₂O and then immersed in 4% paraformaldehyde 4° C. overnight. Lung sections were stained with hematoxylin and eosin (H&E) for histopathology review and morphometric assessment.

Lung Morphometric Assessment Images of the whole left lung section at 20λ amplification were obtained using a eSlide viewer (IMAGESCOPE, Leica Biostystems, Inc., Wetzlar, Germany) and used for assessment as described previously (Ornatowski et al., (2020) Redox Biol 36:101679) to assess the lung morphometric appearance. At this magnification, the entire left lung section was reviewed and evaluated for the emphysematous change, inflammatory cells infiltration, and vascular remodeling. Emphysematous change in the lung was assessed by estimating the air space enlargement using the mean linear intercept measurement (Lm) method (Robbesom et al., (2003) Modern Pathology 16:1-7). For Lm determination, a transparent sheet with ten parallel horizontal lines of equal length and width was superimposed onto scanned H&E histologic images at 20× amplification in IMAGESCOPE eSlide viewer (Leica Biostystems, Inc., Wetzlar, Germany) displayed on a computer screen for respective lung sections. All the image fields free of bronchioles, vessels, collapsed alveoli, and obvious fibrosis within the entire scanning image were counted. On average, about 30-50 image fields were counted per animal. Only the alveolar septa that cross the ten horizontal lines were counted as intercepts. The mean linear intercepts were calculated as the total grid length divided by the average value of the intercepts count per image field per animal. A decreased Lm is expected in the lungs with less air space enlargement and vice versa.

### ATII and ATI In Vitro Cell Model and IncuCyte Apoptosis Assay

For in vitro cell culture system, day 2 culture of freshly isolated rat ATII cells or day 5 culture of freshly isolated rat ATI cells were preincubated with either 3.75 mg/mL or 7.5 mg/mL PEP in calcium-free DMEM medium supplemented with 2% FBS for 24 hours before adding cigarette smoke extract (CSE). As described previously, cigarette smoke extract was prepared from one cigarette in a 10 mL of calcium-free DMEM basal medium. Following preincubation with PEP, cells were stimulated with 2.5% cigarette smoke extract for six hours, and then cell lysate was collected for western blot analysis.

In separate experiments, the role of PEP in reducing cigarette smoke extract-induced apoptotic cell death in cultured ATII and ATI cells was determined in a live cell analysis system (INCUCYTE, Essen Bioscience, Inc., Ann Arbor, MI) as described previously (Zhang et al., (2021) Am J Physiol Lung Cell Mol Physiol 321:L1006-11022). Under the experimental conditions, apoptotic cells were labeled with INCUCYTE Caspase-3/7 green apoptosis reagent (Essen Bioscience, Inc., Ann Arbor, M1, 1:1000 dilution). INCUCYTE NucLight rapid red dye for nuclear labeling (Essen Bioscience, Inc., Ann Arbor, MI, 1:200 dilution) and 2.5% cigarette smoke extract to be tested was mixed with the green apoptosis reagent before adding into the media. Cells were incubated in calcium-free DMEM supplemented with 2% FBS or PEP (3.75 mg/mL or 7.5 mg/mL) for 24 hours prior to adding the CS-extract mixture and apoptosis indicating reagents. Each experimental well's initial cell seeding density was optimized to ensure reaching a confluent monolayer at the assay time. The influence of PEP on cigarette smoke extract-induced cellular apoptosis was then assessed by capturing nuclear-localized green fluorescence every three hours using a 10× objective and a standard scan type. The number of green-fluorescent apoptotic cells per image field was manually counted for each well and analyzed for differences across experimental conditions using analytical software (GraphPad Software. Inc., San Diego, CA).

### Western Blot Analysis and Quantitative PCR

Western blot analysis on lung tissue lysate and cultured ATII and ATI cells was performed to examine important pathway markers expression at protein levels. Protein expression on ATII and ATI cells in response to cigarette smoke extract (CSE) with or without PEP preincubation was measured by western blotting as previously described (Zhang et al., (2019) Am J Physiol Lung Cell Mol Physiol 316:L487-1497). Lung tissue lysate extracted from all the mice across all experimental groups was also examined with this approach. Blotted membranes were exposed using an imaging system (ODYSSEY FC, LI-COR Biosciences, Inc., Lincoln, NE), and images were quantified using IMAGE STUDIO LITE software (version 5.2, LI-COR Biosciences).

qPCR on fresh lung tissue lysate was also performed to examine the gene expression at the transcriptional level. RNA easy columns (Qiagen, Hilden, Germany) were used to extract RNA from tissues, and then cDNA was prepared using RNase H-reverse transcriptase (Invitrogen, Carlsbad, CA) for real-time polymerase chain reaction (RT-PCR). Gene expression was analyzed in triplicates using a commercial kit (SYBR GREENER QPCR SUPERMIX, Invitrogen, Carlsbad, CA). The expression level of each gene was quantified using the threshold cycle (Ct) method normalized for the housekeeping gene β-actin. The primers were synthesized at Integrated DNA Technologies (Coralville, IA). The primers for the qPCR are provided in Table 1.

TABLE 1
Primers

| Target | Primer 1 | Primer 2 |
|---|---|---|
| CXCL12 | GTTGCTTGGGAGGAAACAAAT | CACAAATCCGGCTCCCTAAA |
| | AC (SEQ ID NO: 1) | (SEQ ID NO: 2) |
| CASP3 | CTTGGAACGGTACGCTAAGAA | CACTGACTTGCTCCCATGTA |
| | (SEQ ID NO: 3) | T( SEQ ID NO: 4) |
| CAPS8 | CTGCTGGTCAACTTCCTAGAC | CATGACCCTGTAGGGAGAAA |
| | (SEQ ID NO: 5) | TC (SEQ ID NO: 6) |
| B-actin | CCGTAAAGACCTCTATGCCAA | AGGAGCCAGAGCAGTAATCT |
| | C (SEQ ID NO: 7) | (SEQ ID NO: 8) |

### RNA Sequencing and GSEA and IPA Analysis

FFPE lung tissue sections were processed for next-generation sequencing by HTG Molecular Diagnostics (Tucson, AZ), from which the HTG EdgeSeq mouse immuno-oncology biomarker panel (HTG Molecular Diagnostics, Inc., Tucson, AZ) was chosen to detect a total of 2549 genes quantitatively. The differential expression analysis was completed using the DESeq2 package (Love et al., (2014) Genome Biology 15:550, version 1.12.1) available from Bioconductor. The DESeq2 package provides methods for estimating and testing differential expression using negative binomial generalized linear models. Empirical Bayes methods were used to estimate dispersion and log 2 (fold change) with a data-driven prior distribution.

Normalization selected genes on raw counts data were subjected to gene set enrichment analysis (GSEA, version 4.1.0, Broad Institute) using information from the HALLMARK collection within the Molecular Signatures Database. Generally, the enriched pathway was defined as nominal (NOM) p<0.05, false discovery rate (FDR) q<0.25, and |normalized enrichment score (NES)|>1. To strengthen the credibility of results from the GSEA, genes differentially expressed between 7.5 mg/mL PEP or 15 mg/ml. PEP with cigarette smoke-exposed placebo control mice as well as genes differentially expressed between cigarette smoke-exposed control mice versus room air control mice were extracted by using the DESeq2 analysis algorithm. Following this analysis, a second bioinformatics software package, Ingenuity Pathway Analysis (IPA, Ingenuity System Inc., Redwood City, CA), was used to analyze the differentially expressed genes (fold change>1 and p<0.05); this number was selected as the ideal size for core analysis of gene expression data using the IPA approach. The graphical summary and top canonical pathways were exported to examine altered activities from upstream regulators to signaling pathways involved and the biological functions between the paired group comparisons. The graphical summary provides an overview of the significantly predicted biological entities' overall biofunctions. A Z-score >2 for the activated state and a Z-score<−2 for the inhibited state are generally significant.

### Immunofluorescence Staining of Lung Tissue Section and Live-Cell Imaging of PEP Uptake

Mouse lung FFPE sections were deparaffinized in CITRISOLV (Thermo Fisher Scientific, Inc., Waltham, MA, cat no. 22-143975, Fisher Scientific) and rehydrated in alcohol series. Following a rinse with distilled water, the slides were boiled in preheated 1 mM EDTA for 20 minutes for antigen retrieval. The slides were immersed in 0.2% Triton X-100/PBS solution for five minutes at room temperature for permeabilization. After blocking with 10% NGS/0.2% Triton PBS in a humidified box for 45 minutes, the slides were incubated with various sets of primary antibodies at concentration of 1:100 dilution overnight at 4° C. For the identification of PEP internalized recipient cells, mouse anti-human specific CD63 or a mixture of CD63 and rabbit anti-ProSpc antibodies was carefully selected and applied to the slides. The next day, after rinses with 0.1% PBST, the slides were sequentially incubated with corresponding secondary antibodies for one hour each at room temperature. Following washes with PBS and complete air drying, the slides were mounted with DAPI as a nuclear counterstain.

Representative Images of the Slides were Captured with an Olympus cellSens Dimension System.

DiR fluorescently labeled PEP was added into the ATII or ATI cell culture dish with optical glass-bottom for imaging purposes to examine if PEP can be readily uptake by target cells. These DiR-PEP co-incubated cells were immediately examined for PEP uptake at 15 minutes intervals under the fluorescence microscope using a 60× objective. Snapshot images were captured with an imaging system (CELLSENS DIMENSION, Evident Scientific, Inc., Waltham, MA).

### Lipophilic Dye Exosome Membrane Labeling

Exosomes were labeled with a lipophilic dye to facilitate preliminary evaluation of tissue uptake. Here, DiR and DiI were reconstituted in DMSO concentrations. For in vitro labeling, DiI was added to PEP to a final concentration of 5 μM and incubated at 37° C. for 10-20 minutes. Excess dye was removed by centrifugation through 100 kDa Amicon filters at 4000×g for 15 minutes. For in vivo labeling, DiR and DiI were added to PEP to a final concentration of 50 μM and excess dye was removed by centrifugation.

### Porcine Studies

Intravenous injection and nebulization were tested for global pulmonary targeting, while pulmonary artery (PA) balloon catheter-guided delivery was evaluated for local delivery to a subset of lung tissue. Sedation was performed by intramuscular injection of Telazol (5 mg/kg)/Xylazine (1-2 mg/kg) for intubation followed by continuous inhaled isoflurane (1-3%) for the duration of the procedure. Femoral vein access to place a 9 French sheath was performed under ultrasound guidance for intravenous and PA balloon catheter-guided delivery groups. Intravenous treatment with PEP was administered directly through femoral vein sheath over 5 minutes. For PA balloon catheter-guided treatment, Swan Ganz catheter was advanced from femoral vein to pulmonary artery and pulmonary angiogram was performed. A Storq wire was used to direct the catheter to a branch vessel and balloon was wedged. Contrast was injected with angiography to monitor for leak behind the balloon. Once adequate occlusion was achieved, PEP was injected over 5 minutes. Nebulization was performed with a jet nebulizer attached to the endotracheal tube. All animals received 1.5×10¹³ exosomes labeled with DiR far-red dye. Following treatments, animals were maintained on anesthesia for 15 minutes then euthanized with necropsy to collect the heart, lungs, liver, spleen, kidney, esophagus, and trachea for Xenogen IVUS imaging. Following imaging, samples from each organ were fixed in formalin or flash frozen.

### Xenogen IVUS Imaging

At necropsy, tissues were removed and rinsed in saline. DiR was imaged using an excitation wavelength of 745 nm and emission wavelength of 800 nm. DiI was imaged using an excitation wavelength of 535 nm and emission wavelength of 580 nm. Auto exposure was used to set exposure time for each image.

### Histologic Analysis

Tissues were rinsed in saline and fixed in 10% formalin overnight. Tissues were the embedded in paraffin. Immunocytochemistry was performed with immPRESS HRP Horse Anti-Rabbit IgG Polymer Detection Kit (Vector Laboratories, #MP-7401) and IhmPACT DAB (Vector Laboratories, #SK-4105) kits. Briefly, slides were deparaffinized by sequential washes in xylene, 100% ethanol, 95% ethanol, and distilled water. Antigen retrieval was performed with 10 mM sodium citrate buffer pH 6.0 with 0.05% Tween and heated in a pressure cooker for 10 minutes. Sections were then blocked in 2.5% normal horse serum for 20 minutes followed by primary antibody (CD63, 1:500, R&D Systems, #MAB50482) for one hour. ImmPRESS horse reagent was added for 30 minutes followed by DAB solution for four minutes. Slides were counterstained with hematoxylin, cleared from distilled water to xylene, and sealed with a coverslip. Imaging was performed on AxioScan Zi microscope. Image analysis was performed in ImageJ by RGB color thresholding.

### Statistical Analysis

One-way ANOVA analysis with Tukey-Kramer posttest or two-way ANOVA analysis with Bonferroni multiple comparisons were performed using PRISM 9.0.0 software (GraphPad Software, Inc., San Diego, CA) to evaluate the statistical differences among experimental groups. Results with pC 0.05 were considered statistically significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of treating a pulmonary condition in a subject having or at risk of having the pulmonary condition, the method comprising:
administering to the subject a therapeutic composition in an amount effective to treat the pulmonary condition, the therapeutic composition comprising:
a purified exosome product (PEP) exosomes; and
a pharmaceutically acceptable carrier.

2. The method of claim 1, wherein the PEP exosomes are spherical or spheroid exosomes having a diameter no greater than 300 nm.

3. The method of claim 1, wherein the PEP exosomes are spherical or spheroid exosomes having a mean diameter of 110 nm±90 nm.

4. The method of claim 3, wherein the PEP exosomes are spherical or spheroid exosomes having a mean diameter of 110 nm±50 nm.

5. The method of claim 4, wherein the PEP exosomes are spherical or spheroid exosomes having a mean diameter of 110 nm±30 nm.

6. The method of claim 1, wherein the composition comprises from 1×10¹⁰ PEP exosomes to 1×10¹¹ PEP exosomes.

7. The method of claim 6, wherein the composition comprises from 1.25×10¹⁰ PEP exosomes to 5×10¹⁰ PEP exosomes.

8. The method of claim 7, wherein the composition comprises from 2.5×10¹⁰ PEP exosomes to 5×10¹⁰ PEP exosomes.

9. The method of claim 1, wherein the therapeutic composition is formulated for delivery to at least a portion of the subject's pulmonary tract.

10. The method of claim 9, wherein the therapeutic composition is formulated for delivery to at least a portion of the subject's lung bed.

11. The method of claim 9 or claim 10, wherein the therapeutic composition is nebulized.

12. The method of claim 1, wherein the therapeutic composition is administered in an amount effective to decrease lung compliance (Cst), decrease mean linear intercept (Lm), increase lymphocytic infiltrates in perivascular areas, increase proportion of CD4⁺FOXP3⁺ Tregs in perivascular lymphocytes, increase proportion of CD22⁺ plasma cells in perivascular lymphocytes, decrease expression of inflammatory signaling pathway genes, decrease expression of fibrotic signaling genes, decrease expression of PD1/PD-L1 cancer immunotherapy genes, decrease expression of tumor microenvironment pathway genes, decrease phagosome formation, decrease expression of tumor necrosis factor (TNF), decrease expression of IL-17A-regulated pathways, decrease immune cell chemotaxis, decrease expression of NF-κB signaling pathway genes, decrease expression of senescence pathway genes, increase expression of wound healing pathway genes, decrease S100A8⁺ macrophages in alveolar and/or interstitial regions of the lung, decrease S100A9⁺ macrophages in alveolar and/or interstitial regions of the lung, increase heme oxygenase-1 (HO-1) in a whole lung lysate, decrease NF-κB p65 in a whole lung lysate, decrease caspase 8 in a whole lung lysate, decrease caspase 3 in a whole lung lysate, decrease CXCL12 transcription, increase expression of an antioxidant by alveolar epithelial cells, decrease apoptosis of alveolar epithelial cells, or any combination of two or more of the foregoing, compared to a subject to which the PEP composition is not administered.

13. The method of claim 12, wherein the antioxidant comprises SOD 1-3 or heme oxygenase (HO-1).

14. The method of claim 1, wherein the PEP exosomes contain an antioxidant compound.

15. The method of claim 14, wherein the antioxidant compound is heme oxygenase (HO-1).

16. The method of claim 1, wherein at least a portion of the PEP exosomes are modified to include at least exogenous active agent.

17. The method of claim 16, wherein the exogenous active agent comprises a polypeptide or a nucleic acid.

18. The method of claim 17, wherein the nucleic acid is an mRNA that encodes a therapeutic polypeptide or an inhibitory RNA.
